(19) United States
(12) Patent Application Publication
(10) Pub. No.: US 2025/0090055 A1
(43) Pub. Date: Mar. 20, 2025

(54) METHOD AND DEVICE FOR DETERMINING ISCHEMIC INJURIES OF MAMMALIAN ORGANS AND TISSUES

(71) Applicants: Alexandre Ivachtchenko, Hallandale, FL (US); Andrey Ivashchenko, Hallandale, FL (US); Nikolay Savchuk, Rancho Santa Fe, CA (US); Valentin Volgushev, Hallandale, FL (US); Nikolai Merkin, Hallandale, FL (US)

(72) Inventors: Alexandre IVACHTCHENKO, Hallandale, FL (US); Andrey IVASHCHENKO, Hallandale, FL (US); Nikolay SAVCHUK, Rancho Santa Fe, CA (US); Valentin VOLGUSHEV, Hallandale, FL (US); Nikolai MERKIN, Hallandale, FL (US); Mikhail SLOTVITSKY, Hallandale, FL (US); Andrey BEREZHNOY, Hallandale, FL (US); Valeriya TSVELAYA, Hallandale, FL (US); Konstantin AGLADZE, Hallandale, FL (US); Aleria AITOVA, Hallandale, FL (US); Serafima SCHERBINA, Hallandale, FL (US)

(73) Assignees: Alexandre Ivachtchenko, Hallandale, FL (US); Andrey Ivashchenko, Hallandale, FL (US); Nikolay Savchuk, Rancho Santa Fe, CA (US); Valentin Volgushev, Hallandale, FL (US); Nikolai Merkin, Hallandale, FL (US)

(21) Appl. No.: 18/470,740

(22) Filed: Sep. 20, 2023

(51) Int. Cl. A61B 5/1455; A61B 5/00; A61B 5/145

(52) U.S. Cl. CPC A61B 5/1455; A61B 5/14546; A61B 5/7203; A61B 5/7264

(57) ABSTRACT

The invention relates to medicine, particularly clinical medicine, and can be used in medical diagnostics during surgical procedures on organs and tissues or when preserving an organ to assess its functional state and identify functional disorders. The method and the device implementing it both provide an evaluation by a non-invasive method. In some embodiments, the evaluated organ is affected by NADH-exciting radiation with wavelengths in the near ultraviolet range or the visible spectrum, followed by registration of the returned fluorescence, conversion of fluorescent data into digital form, and obtaining an assessment of ischemic damage to the organ tissue.

### FIELD OF THE INVENTION

The subject matter of the present application relates to the medical field and enables one to determine the degree and localization of ischemic damage to a mammalian organ and tissue by evaluating changes in the fluorescence intensity of the reduced form of nicotinamide adenine dinucleotide.

### BACKGROUND AND SUMMARY

An increased concentration of nicotinamide adenine dinucleotide (NADH) in tissues (and hence an increased intensity of NADH fluorescence) is a known marker of metabolic and ischemic disorders indicating tissue ischemia, i.e., a lack of oxygen supply to the tissue [H. N. Xu, L. Z. Li [symbol] Quantitative redox imaging biomarkers for studying tissue metabolic state and its heterogeneity. J. Innov. Opt. Health Sci. 2014, 7 (2), 1430002; doi: 10.1142/S179354581430002X. A. M. Wengrowski et al. NADH changes during hypoxia, ischemia, and increased work differ between isolated heart preparations. Am J Physiol Heart Circ Physiol 2014, 306, H529-H537; doi: 10.1152/ajpheart.00696.2013.].

Several approaches for determining tissue ischemia are common. A physician may administer a blood test to check for substances released into the bloodstream when the heart or other organs are damaged. For example, in heart ischemia, tests might be performed to measure levels of cardiac troponin released when heart muscle cells die.

Additionally, ischemia in various organs can often be visualized using different imaging modalities, including (1) Electrocardiogram (ECG): Used to check for signs of heart ischemia; (2) Computed Tomography (CT) scans, Magnetic Resonance Imaging (MRI), Ultrasound: These can help identify areas with reduced blood flow in organs like the brain (in stroke), kidneys, liver, etc.; (3) Angiogram: A contrast dye and X-rays can be used to visualize blockages in arteries; (4) Stress Test: For heart-related ischemia, a patient might undergo a stress test, where heart function is evaluated under stress (usually exercise).

In some cases, a tissue biopsy may be undertaken to determine ischemia, or a physician may administer functional tests to check the performance of a potentially affected organ to determine the extent of the ischemic impact. For example, kidney function tests may be done in the case of renal ischemia.

However, none of the above approaches can inexpensively and non-invasively assess ischemia in real-time. Thus, the present application presents innovative methods and devices for inexpensively and non-invasively determining real-time ischemic damage to tissues, organs, or their areas. In preferred embodiments, these devices and methods are based on the nature of the change in NADH fluorescence profile of tissues over time under the influence of exciting radiation.

Further objects, features, and advantages of the present application will become apparent from the detailed description of preferred embodiments, which is set forth below when considered together with the figures of drawing.

### BRIEF DESCRIPTION OF THE DRAWINGS

FIG. 1 depicts an embodiment of photobleaching curves in one pixel of the heart (Source No. 1, Method No. 1), black (A and C)—photobleaching curves before ischemia, gray (B and D)—photobleaching curves after 3.5 hours of ischemia: (A) before ischemia; (B) after 3.5 hours of ischemia; (C) before ischemia, smoothed curve after noise reduction; (D) after 3.5 hours of ischemia, smoothed curve after noise reduction. The change in the intensity of NADH fluorescence over time for the tissue before and after ischemia has a different character—for tissue after exposure to ischemia, it is more concave and with a more significant difference in the intensity of NADH fluorescence. At the same time, the initial level of NADH fluorescence intensity for tissue after exposure to ischemia is significantly higher.

FIG. 2 depicts a rat heart (Method No. 1, Source 1): (A) before ischemia; (B) after 3.5 hours of ischemia. The increased concentration of NADH, which characterizes ischemic damage, is displayed in a light tone. The method demonstrates that the rat's heart received ischemic damage after 3.5 hours of ischemia.

FIG. 3 depicts a graph of ischemia being monitored over time while a pig donor heart is perfused in an experiment. (Method No. 1-gray, Method No. 2-black, Source No. 1). perfusion stops from 12:00 to 12:10 and from 14:00 to 14:35 (vertical dotted lines). When conditions for intentional ischemia were created (perfusion stop), the method demonstrated an increase in ischemia (NADH concentration). After the resumption of perfusion, ischemia (NADH concentration) decreases.

FIG. 4 depicts a schematic diagram of the method and device for its implementation according to some embodiments of the present application. The main stages of the method for determining ischemic damage to a mammalian organ or tissue and the device blocks for implementing this method are given.

FIG. 5 depicts devices for non-invasive monitoring of the ischemic state of the organ according to some embodiments of the present application, including: (A) Laboratory device; and (B) Portable device. The laboratory device includes: (a) a UV-LED control unit, (b) a UV-LED source of exciting radiation, (c) a lens for focusing, (d) a stand for mounting a UV diode (UV-LED), (e) the object under study, (f) an optical filter, (g) OGME-PZ microscope, (i) camera mounting rack, (k) camera, (l) cable, (m) computer. The portable device includes: (a) a UV-LED control unit, (b) a UV-LED panel, an exciting radiation source, (c) a lens for focusing, (d) the object under study, (e) an optical filter, (f) a camera, (g) a cable, (i) a computer. FIGS. 5A and 5B also depict the object under study.

FIG. 6 depicts typical values of the fluorescence brightness for the selected pixel (along the vertical axis) and the frame number (along the horizontal axis) during photobleaching. The fluorescence brightness values increase from ˜500 conventional units (while the fluorescence excitation source turned off) to 5 800-5 500 conventional units (while the fluorescence excitation source turned on during photobleaching). During the photobleaching over time, the fluorescence brightness considerably decreases, but at the end of the photobleaching period, spontaneous fluctuations (noise) exceed the change in amplitude of NADH fluorescence brightness.

FIG. 7 depicts a block diagram of Methods No. 1 and No. 2, including image processing steps used to obtain a map of ischemic injuries. [SGN]

FIG. 8 depicts an illustration of the steps of processing images of registered fluorescence to obtain a map of ischemic injuries according to method No. 1:
(A) The standard deviation map for the SIGNAL segment;
(B) The standard deviation map for the NOISE segment;
(C) A noise-free standard deviation map;
(D) Map of minimum intensities (Imin) for the SIGNAL segment;
(E) Map of minimum intensities (Imin) for the NOISE segment;
(F) A noise-free map for minimum intensities (Imin);
(G) Map of Imin^{\hat{ }4}/k values;
(H) The final NADH photobleaching map (photobleaching SD map, adjusted to the Imin^{\hat{ }4} map).

FIG. 9 depicts a comparison of the evaluation steps for recorded fluorescence to obtain an ischemic injury map according to Method No. 1 for two consecutive photobleaching records of a single rat heart.
(A) Signal from the same region of the heart for two consecutive photobleaching recordings;
(B) and (C) Calculated maximum intensities for two consecutive photo-bleaching recordings;
(D) and (E) Calculated minimum intensities for two consecutive photo-bleaching recordings;
(F) and (G) Calculated Standard deviations for two consecutive photo-bleaching recordings;
(H) and (I) Calculated values of ischemic injury according to Method No. 1 for two consecutive photobleaching records;
(J), (K) Calculated values of ischemic injury according to Method No. 2 for two consecutive photobleaching records.

For two consecutive photobleaching records, NADH decreases as it is photobleached, while the pause between exposures of exciting radiation increases NADH concentration (FIG. 9A). FIGS. 9B to 9D show no visual difference between the maximum (B and C) and minimum (D and E) fluorescence intensity values during two consecutive photo-bleaching intervals, although the level of NADH concentration differs significantly. Further processing allows one to visualize this difference. Thus, FIGS. 9F and 9K illustrate the visual difference in NADH concentration due to taking into account the pattern of the photobleaching curve.

FIG. 10 depicts a comparison of the processing steps for recorded fluorescence to obtain maps of ischemic injuries according to Method No. 1 and No. 2 for the same recording: (rat heart, same recording)):
Maps of maximum intensities: Method 1 (A) and Method 2 (B);
Maps of minimum intensities: Method 1 (C) and Method 2 (D);
Noise-free standard deviation maps: Method 1 (E) and Method 2 (F);
Standard deviation correction maps: Method 1 (G) and Method 2 (H);
Final ischemic injuries maps: Method 1 (I) and Method 2 (J);

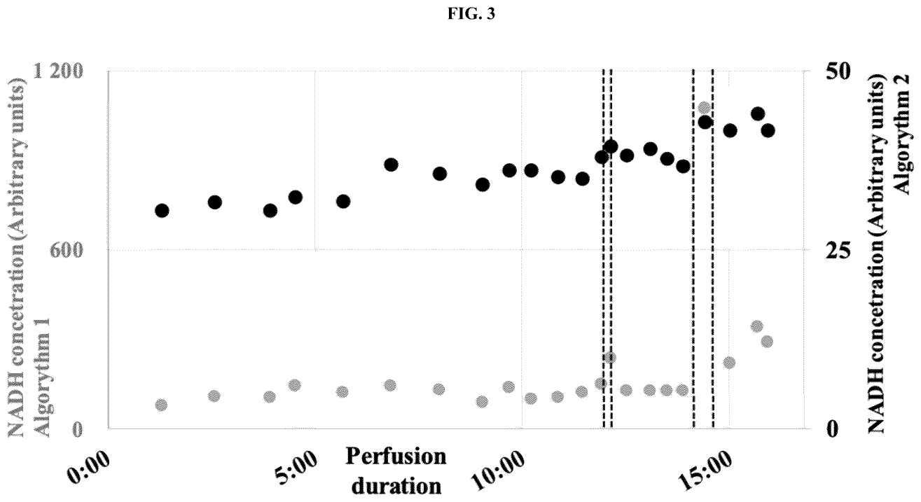

Method No. 1 and Method No. 2 make it possible to identify ischemic injuries due to signal processing. Method No. 1 visualizes ischemic injuries with sharper contrast (more light tones) and demonstrates greater sensitivity. On the other hand, during dynamic observation, Method No. 2 shows a smoother change in NADH concentration, which can be seen in FIG. 3 during exposure to intentional ischemia while preserving an isolated pig heart.

FIG. 11 depicts the performance of a model that describes the chemical kinetics of the NADH photobleaching reaction by solving a system of differential equations to approximate photobleaching curves (continuous lines-experimental data, dotted lines-mathematical modeling): (A) continuous illumination; (B) intermittent pulse illumination. The model of the chemical kinetics of the NADH photobleaching reaction demonstrates quite adequate results both with a single continuous illumination and with intermittent illumination, including working out the restoration of NADH in the interval between pulses.

FIG. 12 depicts an embodiment of constructing a photobleaching map by approximating NADH photobleaching curves by solving a system of differential equations (Source 1): The original images of NADH fluorescence at illumination power of (A) 60% of maximum power, (B) 100% of maximum power; Maps of ischemic damage (NADH concentration) at illumination power of (C) 60% of maximum power; (D) 100% of maximum power. With a higher illumination power (FIG. 12B), there are more light tones in the original NADH fluorescence image than with a lower illumination power (FIG. 12A). However, after modeling, the results of visualization of NADH concentration are visually similar (FIGS. 12C and 12D). Thus, the method of approximation of NADH photobleaching curves by solving a system of differential equations makes it possible to neutralize the influence of the power of the initial illumination to estimate the concentration of NADH.

FIG. 13 depicts an illustration of neural network training results/an error in determining the concentration of NADH in accordance with the number of learning iterations in order to classify photobleaching curves also obtained using a system of differential equations describing the chemical kinetics of NADH photobleaching in tissues: (A) Evaluation loss analysis method; (B) Train loss analysis method; (C) Evaluation score analysis method. All three analysis methods demonstrate successful learning of the neural network and the capability to reach acceptable levels of accuracy in determining the concentration of NADH.

FIG. 14 depicts preprocessing of experimental data for a neural network at 80%, 90%, and 100% of illumination power (A)—Graphs from the same region of the object tissue obtained at 80, 90, and 100% of illumination power; (B)—Same graphs smoothed with a Gaussian filter to reduce noise. Noisy experimental NADH photobleaching curves are smoothed before processing.

FIG. 15 depicts a comparison of different methods to process photobleaching curves (Method 1, Method 2, Differential equations, Neural network) in three consecutive recordings of one organ (human cardiac surgery, image 1 corresponds to the period immediately after the introduction of cardioplegia; image 2, immediately before blood flow restoration; and image 3, immediately after blood flow restoration, before resumed contractions). All four methods (Method 1, Method 2, Differential equations, Neural Network) adequately reflect the increase in ischemia in the tissue after introducing the heart into cardioplegia and improving its condition after the restoration of blood flow.

FIG. 16 depicts plots of NADH monitoring of ischemia in a series of experiments with rat hearts perfused with (A) blood cardioplegic solution and (B) crystalloid cardioplegic solution. In experiments on perfusion of rat hearts in experiments No. 1 and No. 6 with crystalloid cardioplegic solution (FIG. 16B), the value of the conditional NADH concentration exceeded the baseline by more than 2 times, and the heart did not resume contractility at the end of the experiments. In experiments 5 and 7, the baseline was exceeded by less than 2 times during the experiments, and the heart resumed contractility. A similar result may be seen in FIG. 16A for perfusion with blood cardioplegic solution, the heart did not resume contractions in experiment No. 3. Thus, it is shown that for the rat heart, an increase in NADH concentration by 2 times or more indicates significant ischemic damage, leading to the inability to restore the full contractility of the heart.

FIG. 17 depicts ischemic injury maps based on NADH concentration in rat heart conservation experiments: top row (A) to (V) at the beginning of the experiment; middle row (B) to (W) in the middle of the experiment; bottom row (C) to (X) at the end of the experiment. Maps of NADH concentration in experiments on rat hearts, experiments 1-8. The scale (on the right) is plotted from the minimum concentration (in black) to the maximum concentration (in white) for all 8 experiments. In experiments 1, 3, and 6, the heart did not restore contractility, which is illustrated by an increase in the light tones of the brightness of the pictures from top to bottom and corresponds to graphs in FIG. 16 and in Table 2.

FIG. 18 depicts maps of ischemic injuries in rat liver during hypothermic preservation in PBS solution and crystalloid solution: (A) in 1 hour, preserved in PBS; (B) in 24 hours after harvesting, preserved in PBS; (C) in 1 hour, preserved in crystalloid solution; (D) in 5 hours, preserved in crystalloid solution; (E) in 24 hours, preserved in crystalloid solution. After 24 hours of hypothermic preservation in PBS solution (FIG. 18B) and in crystalloid solution (FIG. 18E), ischemic liver damage has increased, visualized by increased bright tones on NADH concentration maps. While some increase of darker tones was registered after five hours of preservation in crystalloid solution (FIG. 18D), that may be associated with metabolism slowdown under hypothermia and, as a consequence, not a complete recovery of NADH concentration during this period after its photobleaching after one hour of preservation (FIG. 18C).

FIG. 19 depicts maps of ischemic injuries in rat kidney during preservation in crystalloid cardioplegic solution: (A) immediately after harvesting; (B) in 2 hours after harvesting; (C) in 4 hours after harvesting. After 4 hours of hypothermic preservation, ischemic kidney damage is not recorded. At the same time, a slight darkening on the second and fifth hours of preservation may be associated with a slowdown in metabolism under hypothermia and, as a consequence, not a complete recovery of NADH concentration for the period after the previous illumination.

FIG. 20 depicts maps of ischemic injuries in rat muscle tissue during preservation in a crystalloid cardioplegic solution: (A) after 1 hour and (B) after 24 hours after harvesting. During 24 hours of hypothermic preservation, ischemic muscle tissue damage increases, which is visualized by an increase in brighter areas of the NADH concentration map. The light tones of the NADH concentration maps may indicate high metabolic needs of muscle tissue.

FIG. 21 depicts a map of ischemic foci in an experiment with a monolayer of human cardiomyocytes and a rat heart:
(A) optical mapping of cardiomyocytes before exposure to a crystalloid cardioplegic solution (gray indicates cells conducting a wave of excitation of contractility);
(B) optical mapping of cardiomyocytes after 4 hours of exposure to crystalloid cardioplegic solution and 7 days of preservation in a nutrient medium (gray indicates cells conducting a wave of excitation of contractility);
(C) map of relative NADH concentration in cardiomyocytes (after 4 hours of exposure to crystalloid cardioplegic solution and 7 days of preservation in nutrient medium), Model No. 1, Source 1;
(D) map of relative NADH concentration in cardiomyocytes, in pseudocolors, after referencing NADH and conducting a wave of excitation of contractility. White-NADH concentration above the critical one, dark-NADH concentration that corresponds to an absence of damage;
(E) rat heart in experiment 1-initial value of NADH concentration is 100% of baseline calculated for the whole fluorescent spot according to FIG. 20, but in pseudocolors visualized in FIG. 21D;
(F) rat heart in experiment 1-peak value of NADH concentration is 271% of baseline calculated of the whole fluorescent spot according to FIG. 20, but in pseudocolors visualized in FIG. 21D.

The scale constructed on the basis of optical mapping of the monolayer of human cardiomyocytes and expanded to 3D using a model of the kinetics of the chemical reaction of photobleaching adequately reflected the ischemic damage to the rat heart obtained in experiment 1 of Example 1.

FIGS. 22A, 22B, and 22C depicts monitoring graphs for ischemia in the perfused pig heart:
(A)—Experiment No. 6, Source 1, processing technique: Method No. 1 and Method No. 2;
(B)—Experiment No. 13, Source 1, processing technique: Method No. 1 and Method No. 2;
(C)—Experiment No. 14, Source 2, processing technique: Method No. 1 and Method No. 2.

The concentration of NADH in arbitrary units calculated by Method No. 1 is gray (Y-axis on the left) and black (Y-axis on the right) NADH concentration in arbitrary units calculated according to Method No. 2. In experiment 6 (FIG. 22A), after 10 hours of preservation, undesirable processes began to develop, leading to organ death, illustrated by a very high increase in NADH concentration. The final decrease in concentration may be explained by the fact that the enzyme that restores NADH has stopped working. In experiments 13 and 14 (FIGS. 22B and 22C), the organ was successfully preserved, and the concentration of NADH varied within acceptable limits. Method No. 1 demonstrates greater sensitivity, which explains the significant differences in values. Method No. 2 provides more smooth monitoring of NADH concentration.

FIG. 23 depicts maps of ischemic lesions of the pig heart when using Source 2 in Experiment No. 13: (A) Method No. 1, at the start of the experiment; (B) Method No. 1, middle of the experiment; (C) Method No. 1, completion of the experiment; (D) Method No. 2, beginning of the experiment; (E) Method No. 2, in the of the experiment; (F) Method No. 2, final stage of the experiment. Method No. 1 and Method No. 2 both show an increase in ischemic damage in the perfused heart over time. At the same time, Method No. 1 demonstrates greater sensitivity, which is confirmed by the lighter tones of the NADH concentration maps in the middle and at the end of preservation.

### ABBREVIATIONS, TERMS, AND DEFINITIONS

“NAD” (Nicotinamide Adenine Dinucleotide) is a coenzyme found in all known living cells. NAD is involved in redox reactions in cell metabolism by transferring electrons from one reaction to another. NAD in cells exists in two functional states: its oxidized form, NAD+, is an oxidizer and takes electrons from another molecule, being reduced to NADH, which further serves as a reducing agent and gives away electrons.

“NADH fluorescence” is the luminescence of NADH molecules when exposed to ultraviolet light.

NADH “photobleaching” is the decrease in NADH fluorescence intensity over time when exposed to ultraviolet radiation.

NADH photobleaching “intensity” is the rate at which the NADH fluorescence intensity decreases over time when exposed to ultraviolet light. It is determined by the rate of transition of NADH to other functional states, e.g., NAD, NAD+.

NADH photobleaching “pattern” is a curve characterizing the intensity of NADH photobleaching over time under the influence of ultraviolet radiation. In other words, it is a curve reflecting the character of the change in NADH fluorescence intensity over time. The photobleaching pattern depends on the presence and degree of ischemic damage and can thus serve as a basis for the assessment of ischemic tissue injury.

A photobleaching “map” (map) is a visual image of the organ/tissue under study in the form of a map (diagram, picture, etc.) or an array of digital data of the primary recorded fluorescence or processing results.

A “kinetic model” is a system of differential equations describing a kinetics model for chemical reactions of NADH photobleaching occurring in a tissue. It is used to evaluate NADH concentration in the tissue based on the NADH photobleaching curve.

### DETAILED DESCRIPTION OF THE INVENTION

The first aspect of the invention is a method for determining ischemic damage to organs, parts thereof, or tissues of mammals. In a preferred embodiment, the method includes excitation of NADH fluorescence and its registration, followed by processing or evaluating fluorescence intensity values, which considers the nature (character) of NADH fluorescence change over time.

In a preferred embodiment, a distinctive feature of this method is the subsequent processing of the values of changes in the fluorescence intensity over time after the excitation of NADH fluorescence.

The application of this invention is possible both during surgical interventions on organs and tissues and while preserving a donor organ or tissue to identify tissue and functional disorders, their localization, and determination of their reversibility.

One embodiment of the present invention provides a method that involves refining the registered fluorescence signal to substantially reduce or eliminate noise.

Moreover, the method includes processing the registered fluorescence by determining the standard deviation.

In another embodiment, the application also includes a method that includes processing by dividing the value of the standard deviation of the fluorescence intensity by the value of the minimum intensity of excited fluorescence raised to the fourth power.

In another embodiment, the application provides a method that includes processing by dividing the value of the standard deviation by the value of the minimum intensity of excited fluorescence, raising the result to ⅛ power, and then dividing by the maximum value of fluorescence.

In another embodiment of the present invention, the method includes processing by approximation of photobleaching intensity curves by solving a system of differential equations describing the kinetic model of photobleaching, in order to return the concentration of NADH, the power of exciting radiation and the strength of the NADH reducing enzyme.

In yet another embodiment of the present invention, the method includes processing by classification of photobleaching intensity curves by a neural network, which returns for each curve a unique set of variables, including the concentration of NADH, the power of exciting radiation, and the strength of the NADH reducing enzyme.

The subject of the present invention is also a method in which radiation that excites NADH fluorescence has a wavelength of 365 nm.

The subject of the present invention is also a method in which NADH fluorescence with a wavelength of 460 nm is recorded.

The subject of the present invention is also a method in which the ratio of time and specific power of the exciting radiation should be sufficient to obtain a photobleaching curve.

In accordance with the present invention, the assessment of ischemic damage is carried out repeatedly over time to track the dynamics of the appearance and development of ischemic foci.

The second aspect of the present invention is a device for determining ischemic tissue damage, including a unit for exciting NADH fluorescence, a unit for recording NADH fluorescence, and a unit for processing fluorescence intensity values taking into account the nature of its change over time.

In accordance with the present invention, the device processes the registered fluorescence, and the processing involves refining the registered fluorescence signal to be free from noise.

In one embodiment, the device processes the recorded fluorescence by calculating the standard deviation.

Moreover, the device processes the recorded fluorescence by dividing the value of the standard deviation of the fluorescence intensity by the value of the minimum intensity of the excited fluorescence, raised to the fourth power.

Moreover, the device processes the recorded fluorescence by dividing the value of the standard deviation of the excited fluorescence by the value of the minimum intensity of the excited fluorescence, raising the result to the power of ⅛ and then dividing by the maximum value of fluorescence.

In another embodiment of the present invention, the device processes the recorded fluorescence by approximating the photobleaching intensity curves by solving a system of differential equations describing the kinetic model of photobleaching, in order to return the concentration of NADH, the power of exciting radiation and the strength of the NADH reducing enzyme.

In yet another embodiment of the present invention, the device processes the recorded fluorescence by classifying the photobleaching intensity curves with a neural network, which returns for each curve a unique set of variables, including the concentration of NADH, the power of exciting radiation and the strength of the reducing NADH enzyme.

The subject of the present invention is also a device that excites NADH fluorescence with radiation that has a wavelength of 365 nm.

The subject of the present invention is also a device that registers NADH fluorescence with a wavelength of 460 nm.

In accordance with the present invention, the device separates NADH fluorescence with a wavelength of 460 nm using software before cleaning the digital form of fluorescence from noise.

In accordance with the present invention, the ratio of time and specific power of exciting radiation in the device is sufficient to obtain data to compute a map of ischemic organ damage.

In accordance with the present invention, the specific power of ultraviolet radiation in the device to excite NADH belongs to the range of 1 to 50 mJ/mm².

In accordance with the present invention, NADH is recorded in a fluorescence device by photographing at a speed of 50 frames per second with a resolution of 512 by 512 pixels.

The invention is based on an assessment of the nature of NADH photobleaching, taking into account the influence of the uneven distribution of the specific power of the exciting radiation over the surface of the organ. Routine application of the invention is possible both during surgical interventions on organs and tissues and while preserving a donor organ or tissue to identify tissue and functional disorders, their localization, and determination of their reversibility.

The nature of photobleaching differs for normal and ischemic areas of the organ/tissue by the shape and the intensity decay curve. When assessing the degree of ischemia of large areas of tissue or ischemia the whole organs, the invention includes technical solutions to eliminate the influence of an uneven distribution of the specific power of exciting radiation over the surface of the organ resulting from the geometry of the organ and/or mutual displacements of the source of exciting radiation and the irradiated organ, as well as the elimination of edge effects on the border of the organ, where the illumination differs significantly because of the natural shape of the organ. The result is evaluated quantitatively and can be visualized as a map (images, pictures, diagrams, or digital series) of the organ, highlighting ischemic areas of different degrees of injury. Also, the result of consecutive recordings may be visualized as a curve for the entire organ and/or its site or as a transforming map of the organ that reflects the dynamics of the ischemic area's progression over time.

Obtained information (a map of ischemic injuries) may be presented as data arrays, tables, graphs, images, or controlling signals intended for storage or output to other devices.

The NADH biomarker concentration is assessed by recording, measuring, and processing the signal of the excited fluorescence of NADH (fNADH).

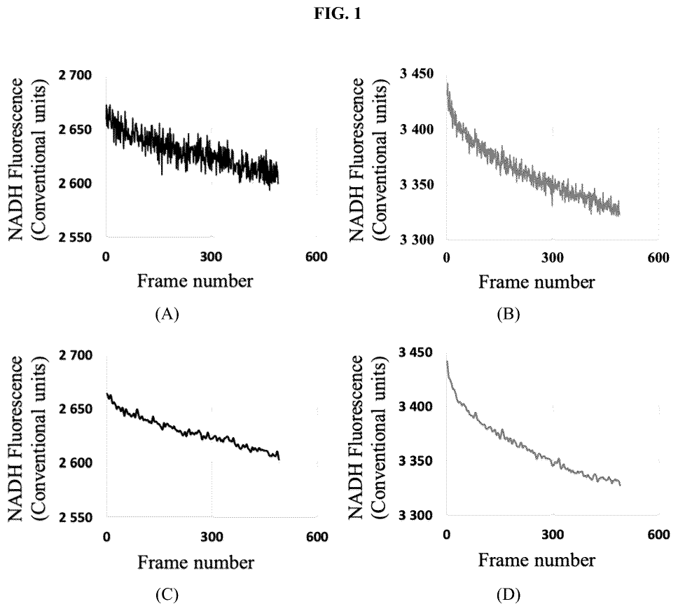

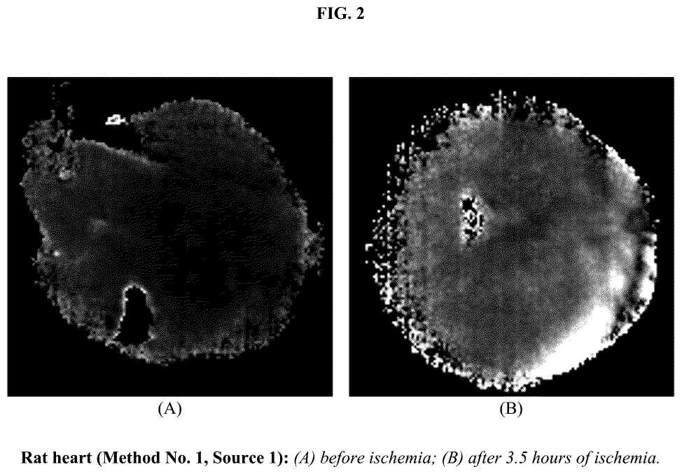

In general, the invention is based on the proposal to use the nature of the photobleaching curve to determine ischemic damage to tissues and organs. The nature of the NADH photobleaching curve depends on the concentration of NADH in the illuminated area. FIG. 1 shows photobleaching curves in the same pixel for the same area of the rat heart at the same illumination power: curve FIG. 1A reflects the nature of photobleaching of a non-ischemic heart; curve FIG. 1B—the nature of photobleaching after 3.5 hours of ischemia. As can be seen, the characters of the curves FIG. 1A and FIG. 1B are different. The nature of the curve, all other things being equal, depends on two parameters—the specific power of the exciting radiation and the concentration of NADH in the illuminated area. If the power of the exciting radiation is the same for two illuminations, then the nature of the photobleaching curve is determined only by the NADH concentration. The invention offers not only the use of the character of NADH photobleaching to determine NADH concentration but also a technical solution for determining the arbitrary concentration of NADH by the nature and character of its photobleaching.

It is shown that only one NADH concentration value corresponds to one photobleaching curve. By determining the arbitrary concentration of NADH in each pixel of the illuminated area of the tissue/organ, it is possible to build a map of NADH concentrations for this area at the time of illumination and thereby assess the absence or presence and degree of ischemia. Repeated illumination of the same area over time makes it possible to observe the dynamics of ischemia progression.

FIG. 2 shows the dynamics of the map of ischemic areas of the same organ at the beginning of ischemia (FIG. 2A) and after 3.5 hours of ischemia (FIG. 2B). The second map depicts a significantly higher level of ischemia (lighter areas) compared to the first map (the beginning of ischemia).

For the convenience of monitoring tissue/organ ischemia over time, it is possible to reduce each illumination recording to a single digital value to represent ischemia progression in the form of a graph, where each point of the graph corresponds to one illumination recording. FIG. 3 shows a graph of ischemia monitoring of a perfused donor pig heart. The method and device of the invention quite adequately reflect the changes in the ischemia of the organ. The first 12 hours of preservation demonstrate an approximately even level of ischemia, while illumination recordings from 12 to 14 hours reflect an increase in ischemia with consequent ischemia decrease. In this case, the graph depicts a 10-minute pause in organ perfusion (an increase in ischemia) at 12:00, followed by a resumption of perfusion at 12:10 (a decrease if ischemia). Illuminations from 14 to 16 hours also indicate an increase in ischemia, followed by a decrease. This part of the graph depicts a 35-minute perfusion pause at 14:00, followed by its resumption at 14:35.

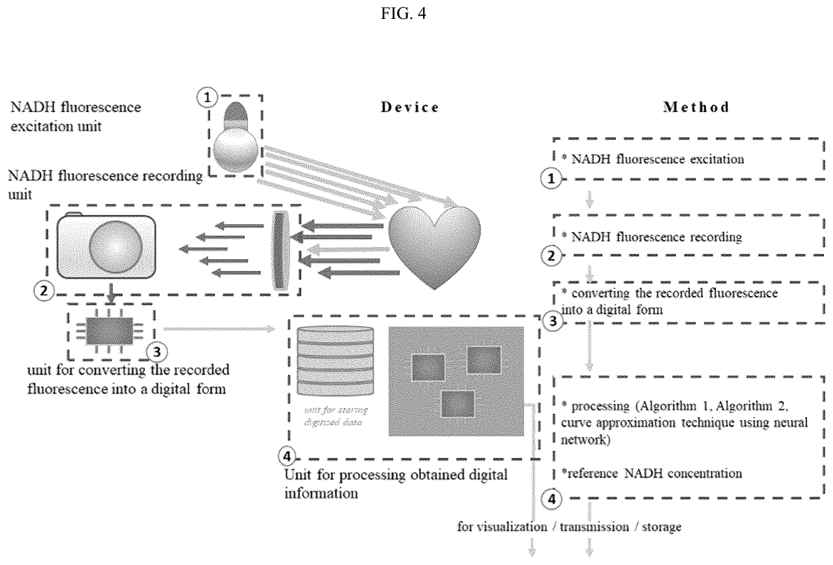

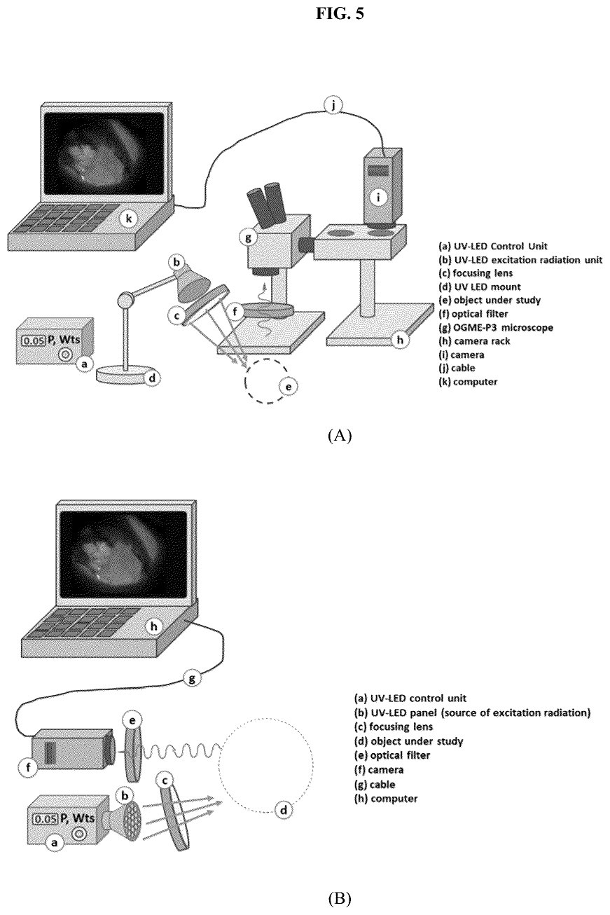

The relationship between the method and the device implementing it is shown in FIG. 4. The device intended to implement the method may be technologically arranged in different ways. For example, the experiments used both a laboratory device (FIG. 5A) with UV LED (wavelength 365±20 nm), a light filter (with a bandwidth of 465±15 nm), and a high-speed camera PCO.1200hs (with a wide range of frame resolution up to 1280×1025 px and shooting speeds up to several thousand frames per second), which transmits a digitized image for further processing on a computer using specialized software, and a portable device (FIG. 5B).

It is also technologically possible to put together a camera and a signal processing unit in one device or to provide a device that combines a camera, a digitized information processing unit, and a device for visualizing a map. A variant of the arrangement of the device with a catheter or probe is allowed, which allows irradiating a small area of the studied tissue with exciting radiation and recording the character of NADH photobleaching in this small area.

### Excitation

Excitation of NADH fluorescence is performed by exciting radiation produced by a light source with a typical wavelength in the range of 320-380 nm [Characterization of NADH fluorescence properties under one-photon excitation with respect to temperature, pH, and binding to lactate dehydrogenase/Taylor M. Cannon, Joao L. Lagarto, Benjamin T. Dyer, Edwin Garcia, Douglas J. Kelly, Nicholas S. Peters, Alexander R. Lyon, Paul M. W. French, and Chris Dunsby/OSA Continuum Vol. 4, Issue 5, pp. 1610-1625 (2021)/https://doi.org/10.1364/OSAC.423082/Received 4 Mar. 2021; accepted 23 Apr. 2021], with the energy of radiation reaching the surface of the test sample required to record photobleaching more than 1 mJ/mm²; in this case, no coherence of the radiation source is required.

According to the experimental results, the energy of radiation reaching the surface of the sample may vary from 1 mJ/mm² (corresponding to an exposure power of 50 mW/cm² for 2 seconds for Source 1) to 50 mJ/mm² (corresponding to an exposure power of 500 mW/cm² for up to 10 seconds for Source 1) for each exposure. According to literature data [Enzyme-dependent fluorescence recovery of NADH after photobleaching to assess dehydrogenase activity of isolated perfused hearts/Angel Moreno, Sarah Kuzmiak-Glancy, Rafael Jaimes 3rd & Matthew W. Kay/nature.com/scientificreports/Scientific Reports 7:45744 DOI: 10.1038/srep45744/received: 30 Sep. 2016/Published: 31 Mar. 2017], the energy of UV radiation that damages the tissue is 42000 mJ/mm² (for an exposure power of 7000 mW/cm² with a duration of 600 seconds for Source 1).

In the experiments on cells, tissues, and small and large mammalian organs, Source 1 (LED with a characteristic wavelength of 365±20 nm and a power of 500 mW/cm² at a distance of 3 cm and a spot diameter of 1.2 cm) was used. In the experiments on large organs (pig and human hearts, in particular), Source 2 was used (a panel of 100 LEDs, 370±15 nm, specific power of about 230 mW/cm² at 3 cm distance with a spot diameter of 7.5 cm). The duration of exposure to exciting radiation varied from 1 to 15 seconds.

In the experiments, the result was achieved using one or more light sources in the form of LEDs with a characteristic wavelength of 365±20 nm and a power of 250 mW, providing a specific illumination power of 25 mJ/mm² (for an exposure of 5 seconds). In addition, the result was achieved using less powerful Source 2 (a panel of 100 LEDs, 370±15 nm, with a specific power of radiation reaching the surface of about 230 mW/cm² at a distance of 3 cm from the light source and a diameter of the spot equals to 7.5 cm, which corresponds to 10-23 mJ/mm² when exposed for 1-15 seconds.

### Recording

NADH fluorescence is recorded for a time interval comparable to the duration of the excitation radiation, which is sufficient to reduce the NADH fluorescence intensity to the extent required to neglect the exponential pattern of photobleaching.

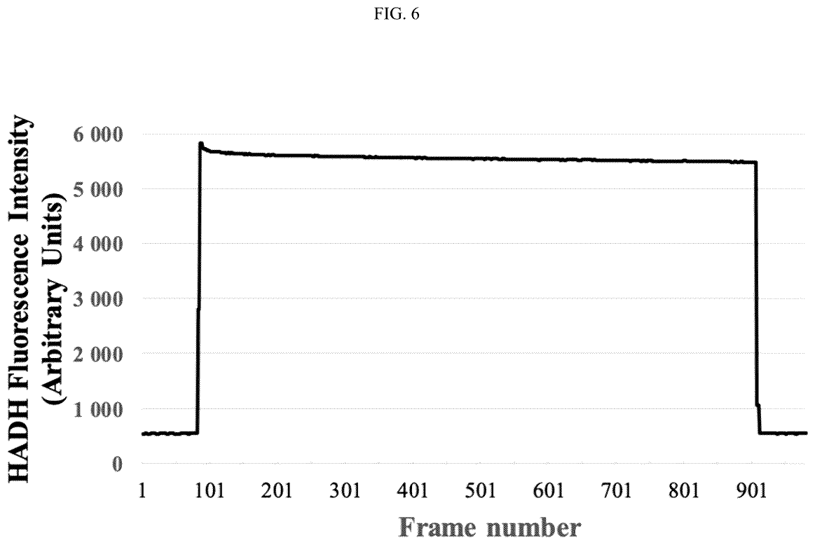

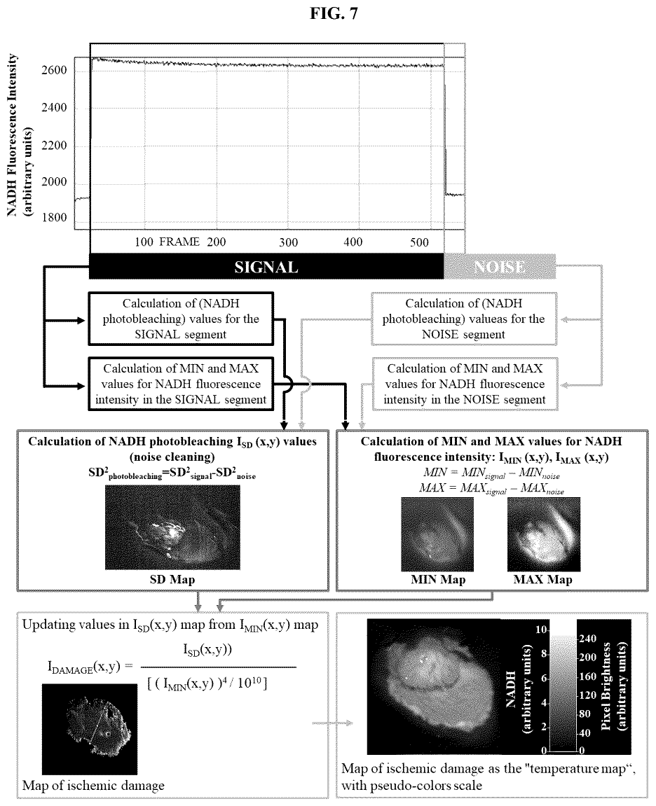

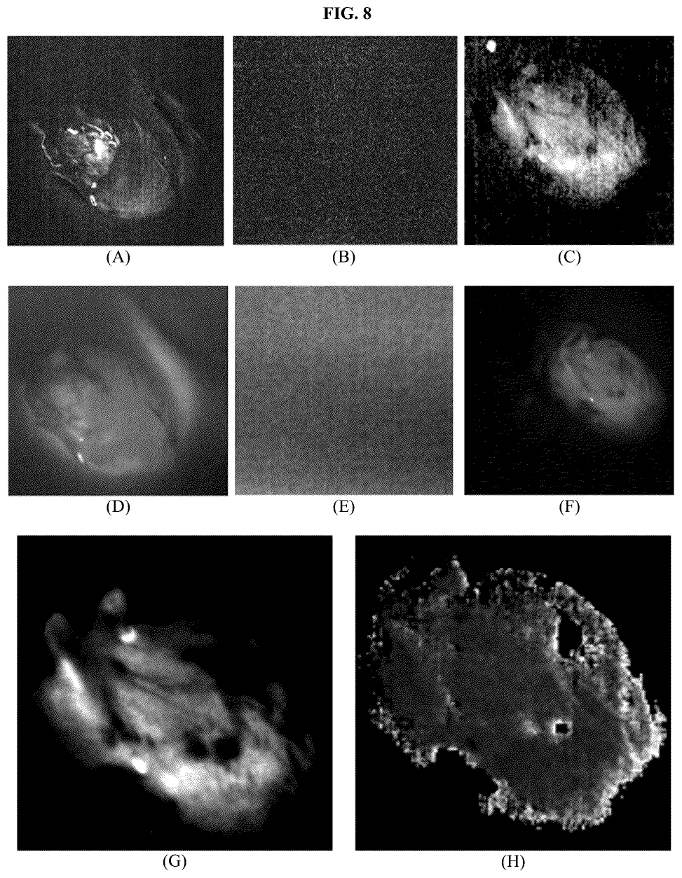

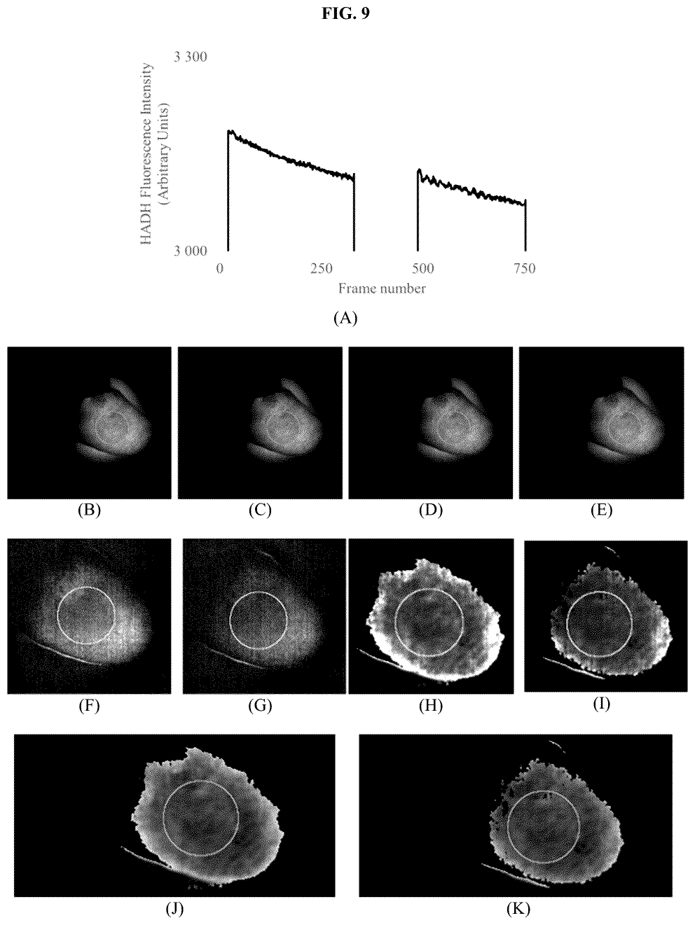

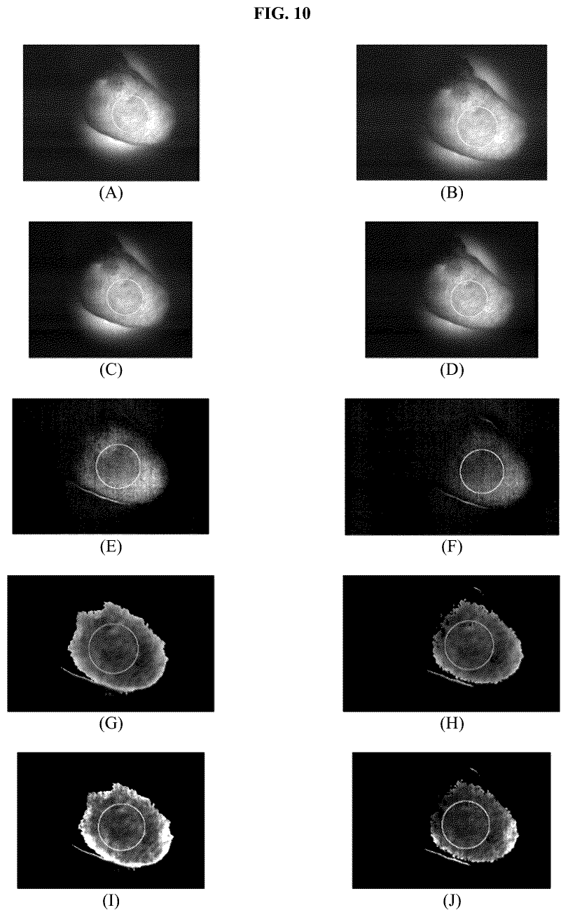

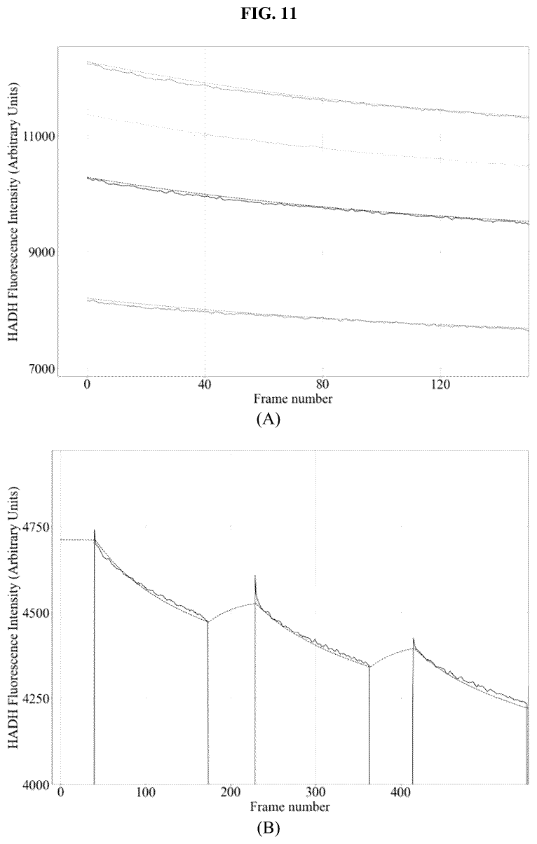

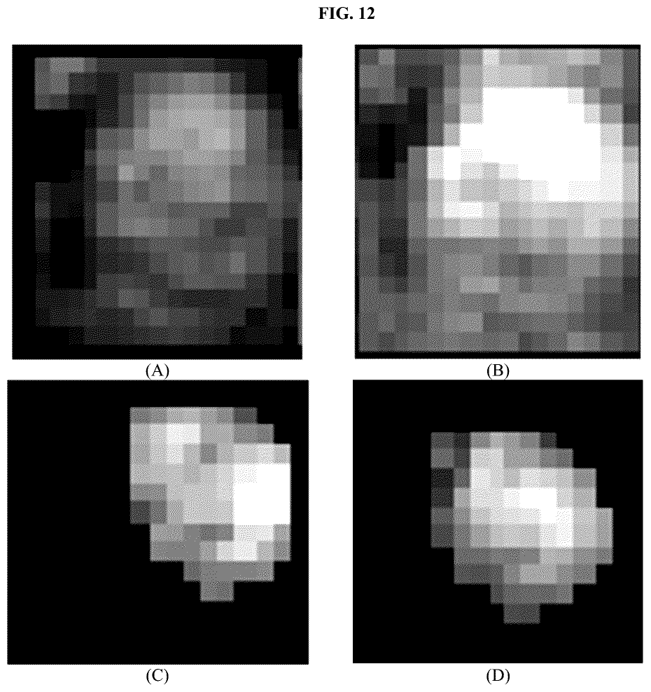

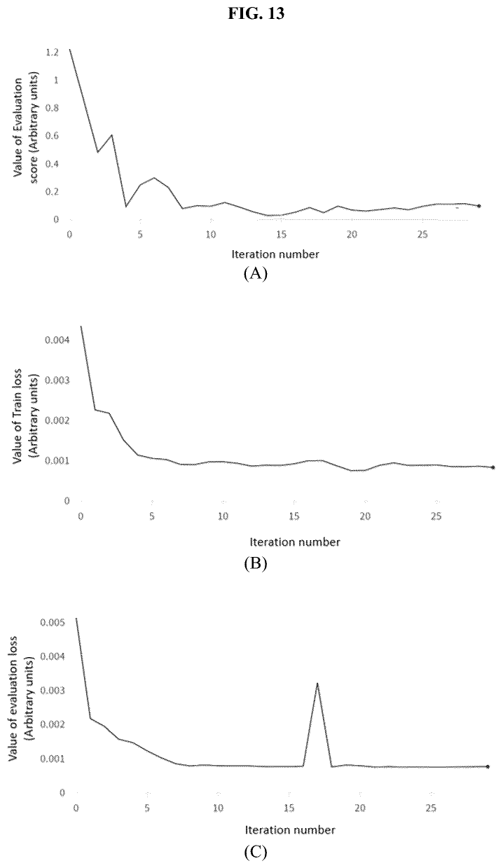

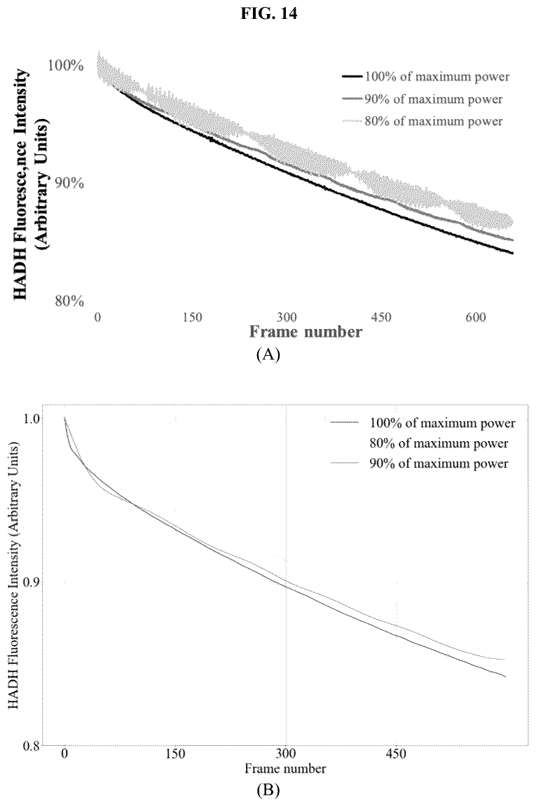

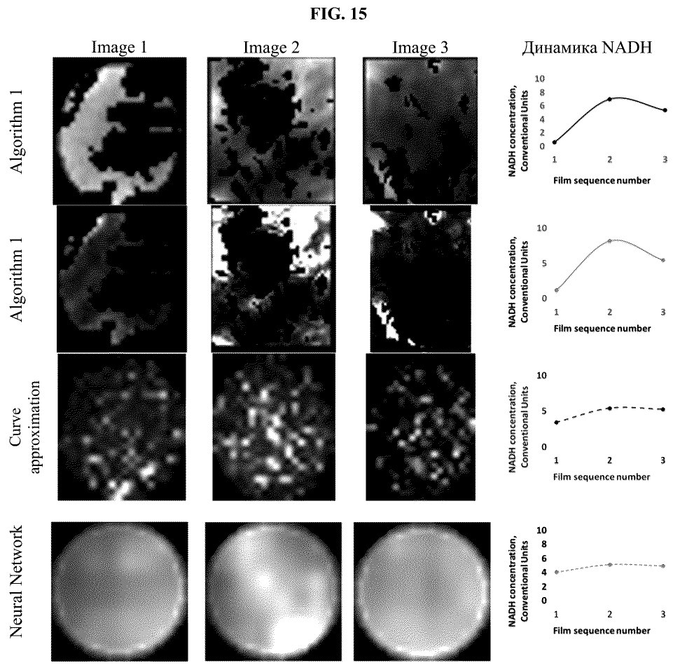

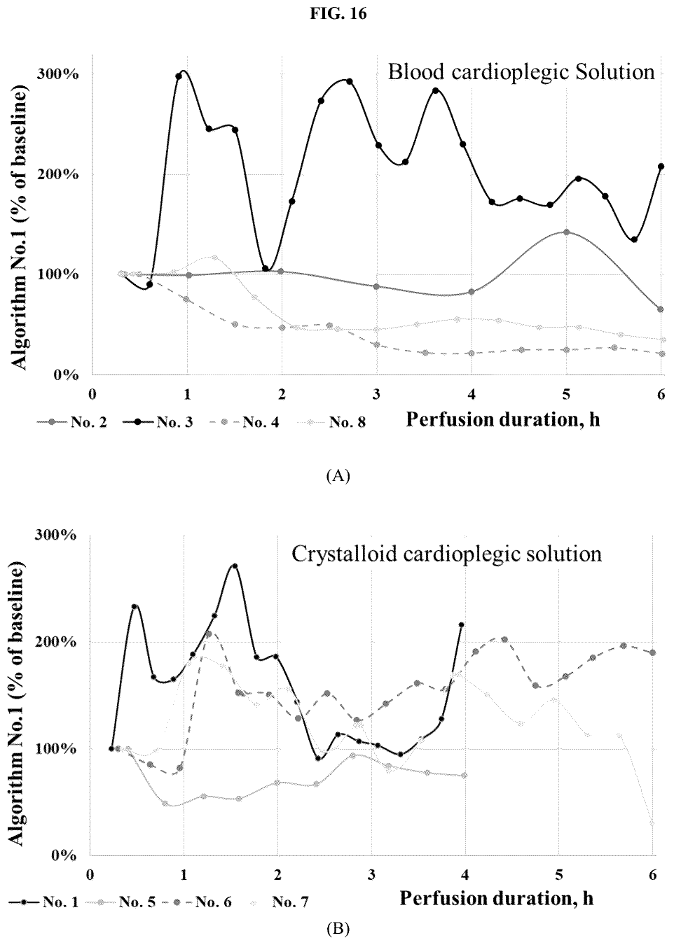

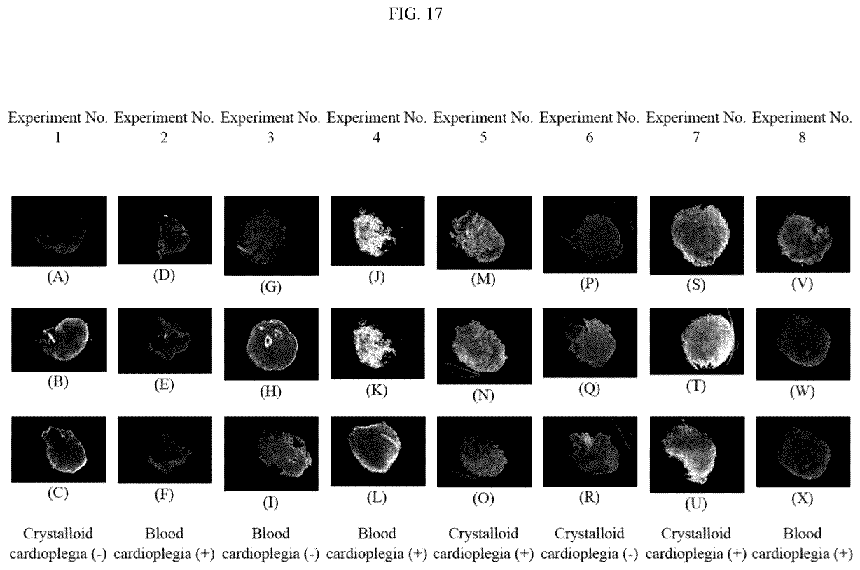

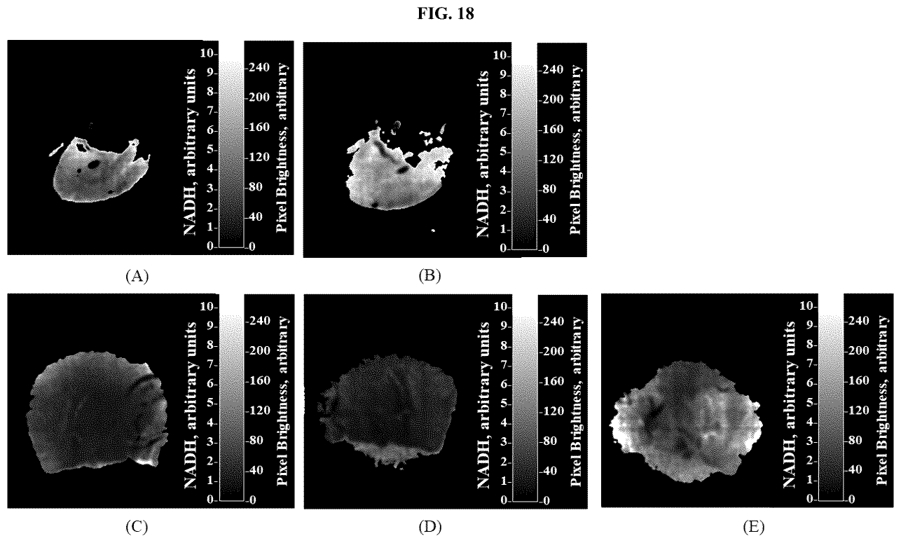

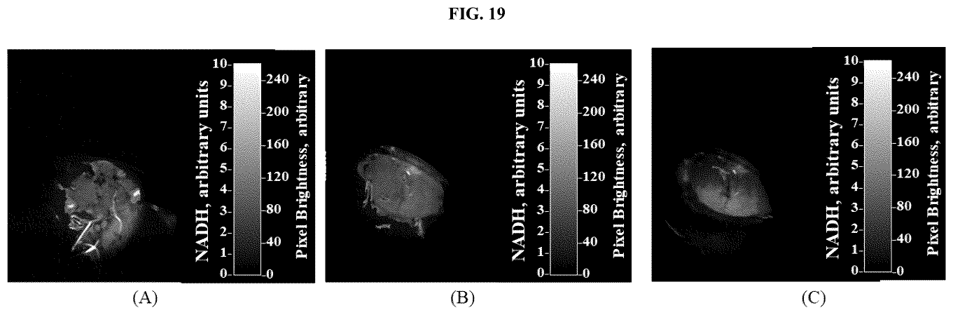

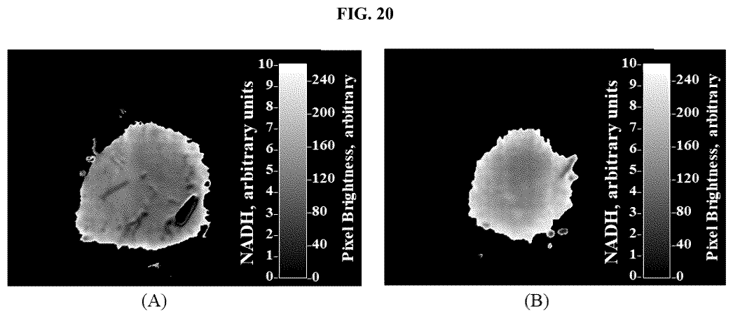

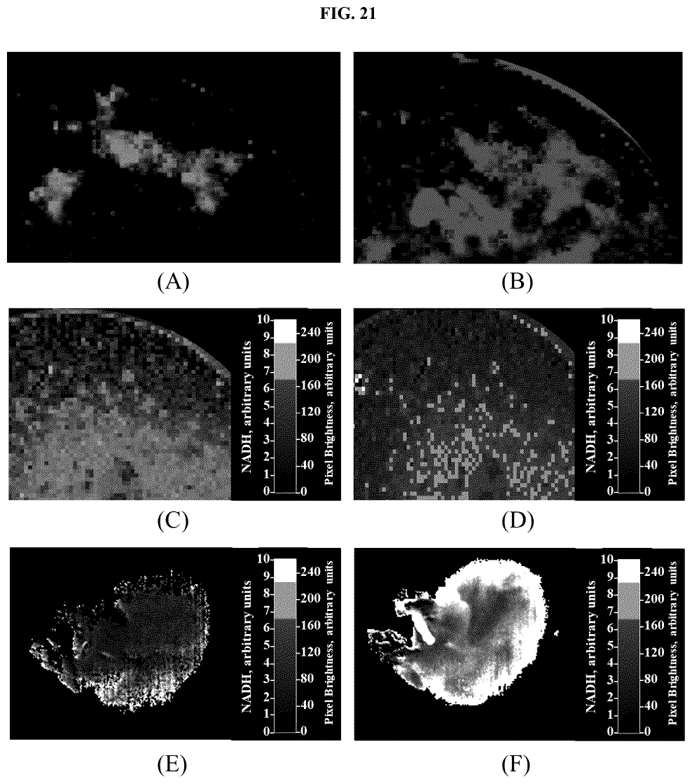

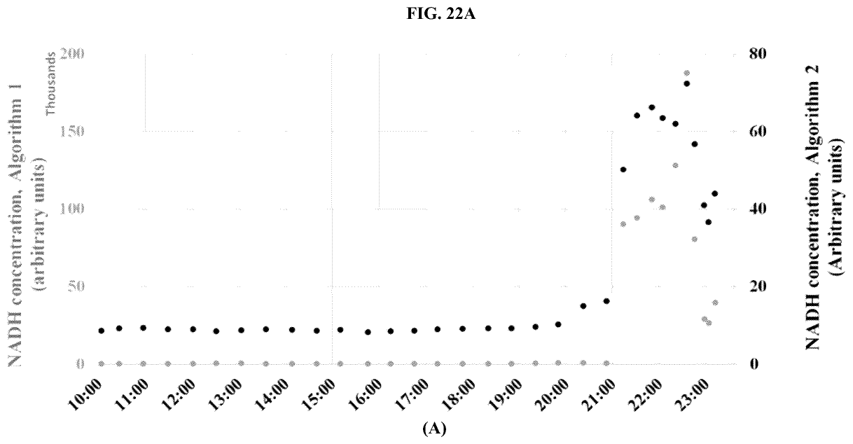

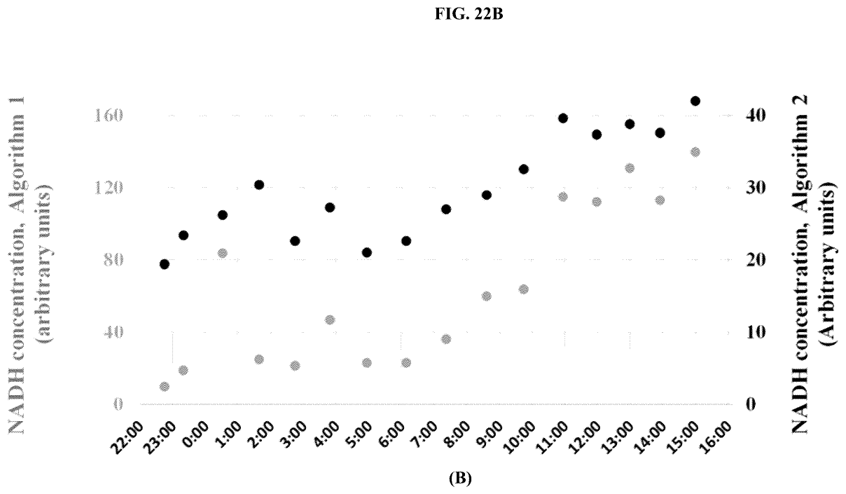

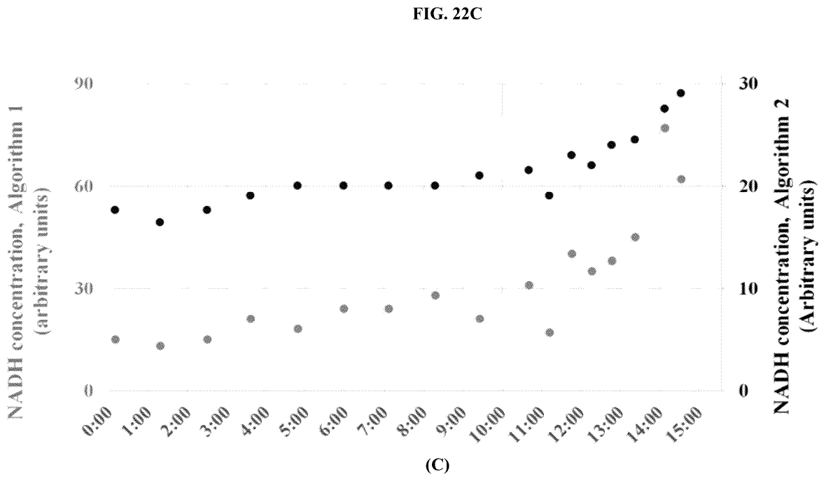

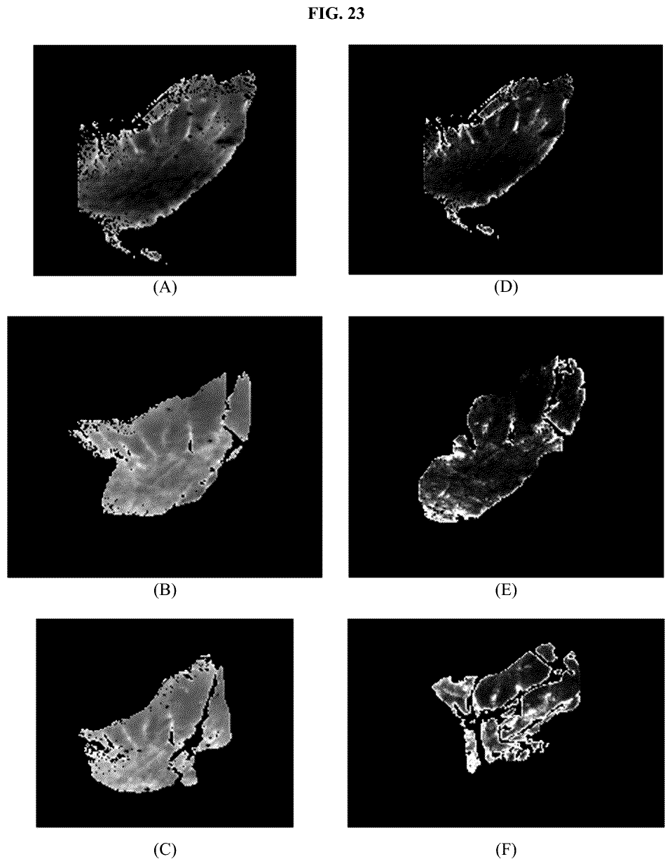

The exponential pattern of photobleaching is related to the fact that under the influence of ultraviolet radiation of sufficient intensity, NADH actively converts to NAD+. The intensity of photobleaching (the rate of decline in fluorescence intensity) decreases over time and approaches or reaches an equilibrium state when the transition of NADH to NAD+ is compensated by the transition of NAD+ to NADH (FIG. 6).

Given the rate of photobleaching depends on the power of exciting radiation and is required to ensure the state of NADH equilibrium is reached, the ratio of time to the specific power of the excitation radiation should be sufficient to obtain data required to assess ischemic organ damage. Exemplary working power-to-time ratios are provided herein.

The effect of photobleaching followed by restoration of NADH fluorescence was studied and described in publications [Enzyme-dependent fluorescence recovery of NADH after photobleaching to assess dehydrogenase activity of isolated perfused hearts/Angel Moreno, Sarah Kuzmiak-Glancy, Rafael Jaimes 3rd & Matthew W. Kay/nature.com/scientificreports/Scientific Reports 7:45744 DOI: 10.1038/srep45744/received: 30 Sep. 2016/Published: 31 Mar. 2017], [Characterization of NADH fluorescence properties under one-photon excitation with respect to temperature, pH, and binding to lactate dehydrogenase/Taylor M. Cannon, Joao L. Lagarto, Benjamin T. Dyer, Edwin Garcia, Douglas J. Kelly, Nicholas S. Peters, Alexander R. Lyon, Paul M. W. French, and Chris Dunsby/OSA Continuum Vol. 4, Issue 5, pp. 1610-1625 (2021)/https://doi.org/10.1364/OSAC.423082/Received 4 Mar. 2021; accepted 23 Apr. 2021], [Direct Imaging of Dehydrogenase Activity within Living Cells Using Enzyme-Dependent Fluorescence Recovery after Photobleaching (ED-FRAP)/C. A. Combs and R. S. Balaban/Biophysical Journal Volume 80 April 2001 2018-2028/Received for publication 2000 Sep. 13/In final form 2001.01.22], [NADH Enzyme-Dependent Fluorescence Recovery after Photobleaching (ED-FRAP): Applications to Enzyme and Mitochondrial Reaction Kinetics, In Vitro/Frederic Joubert, Henry M. Fales, Han Wen, Christian A. Combs, and Robert S. Balaban/Biophysical Journal Volume 86 January 2004 629-645/Submitted Apr. 7, 2003, and accepted for publication Aug. 27, 2003.] The exponential pattern of photobleaching was also studied under different modes of NADH fluorescence excitation [Photobleaching of reduced nicotinamide adenine dinucleotide and the development of highly fluorescent lesions in rat basophilic leukemia cells during multiphoton microscopy/LeAnn M Tiede, Michael G Nichols/Photochem Photobiol. May-June 2006; 82 (3): 656-64. doi: 10.1562/2005-09-19-RA-689/Received 2005.09.15/Accepted 2006.01.18].

In order to obtain the data necessary to eliminate noise in the acquired images, it is also presumed to capture images before and/or after exposure to excitation radiation.

NADH fluorescence is recorded in a wavelength range of 420 to 480 nm [Characterization of NADH fluorescence properties under one-photon excitation with respect to temperature, pH, and binding to lactate dehydrogenase/Taylor M. Cannon, Joao L. Lagarto, Benjamin T. Dyer, Edwin Garcia, Douglas J. Kelly, Nicholas S. Peters, Alexander R. Lyon, Paul M. W. French, and Chris Dunsby/OSA Continuum Vol. 4, Issue 5, pp. 1610-1625 (2021)/https://doi.org/10.1364/OSAC.423082/Received 4 Mar. 2021; accepted 23 Apr. 2021]. Light filters with a bandwidth multiple of this range can be used.

In performed experiments, the result was achieved using a light filter with a bandwidth of 465±15 nm (Olympus B) as well as a narrow bandwidth 460 nm Bandpass Interference Filter: 10 nm FWHM, OD>4.0 Coating Performance with a bandwidth of 460±5 nm.

Recorded NADH fluorescence contains image information over time, including the brightness values of each pixel for each recorded frame (FIG. 6).

The source video may be represented as an array of data containing:
I—intensity values in pixels (16-bit tiff, b&w recording)
x,y—pixel coordinates in the frame (380×351 px is the optimal result in the experiments).
z—frame number (z_max−number of frames in the video=frame rate per second x shooting duration in seconds (491 frames of 380×351 px is the optimal result in the experiments)).

The experiment results were achieved using Recorder No. 1 of the high-speed video camera PCO.1200hs with a shooting speed of up to 400 fps (frames per second) at a resolution of 1280×1024 px. For an optical system, an OGME-PZ microscope (f=190 mm) was used, from which the image was transmitted to the camera via a stand (adapter) for fixing the TV-A camera (FIG. 5A).

This result was also achieved by using Recorder No. 2 (FIG. 5B), an IDS (Imaging Development System GmbH) UI-5220SE-M-GL Rev.2 digital camera with a frame resolution of 752×480 px, shooting speed up to 100 frames per second, and an AZURE-0614 MM lens with a focal length of f=6 mm and a minimum focus distance of 0.1 m as the optical system.

It is acceptable to use equipment that produces and records higher quality images (24, 25, 30 fps for 1440p (2560×1440 px), 48, 50, 60, 120 fps for 1080p (1920×1080 px) and 240 fps for 720p (1280×720 px) ISO (50 to 3200 and higher).

The result in the above experiments was achieved with an image captured at 49 frames per second at a resolution of 380×351 px and a color depth of 16-bit.

The minimum recommended image settings for shooting are a resolution of 256×256 px and a shooting speed of 25 fps.

The results described in the examples were achieved in the experiments when the images were converted to TIFF (16-bit) format with a constant discrete time step (sampling frequency). However, in some applications, it is acceptable to use formats in which the image is encoded using any common video encoding standard [https://en.wikipedia.org/wiki/Video_coding_format].

The image is digitally recorded on the in-camera media and/or transferred to an external source for storage and further processing.

### Processing

The purpose of the processing is to obtain an NADH concentration value from the photobleaching intensity in each pixel and in the whole illuminated area in order to assess the degree of tissue ischemia and to exclude the influence of non-uniform exposure to excitation radiation due to the properties of the radiation source and/or shape of the organ/tissue, including marginal effects. In this case, the value of the NADH concentration is determined by the photobleaching pattern (the shape of the photobleaching curve). Generally, the determination of the NADH concentration value is based on the statement that one photobleaching curve corresponds to one NADH concentration value. In other words, all NADH photobleaching curves are unique, and two NADH concentration values cannot produce the same curve.

The resulting initial image undergoes primary frame-by-frame processing that includes but is not limited to, the following steps: increase the color depth to 32-bit, smooth the image over time and space (e.g., Gaussian blur), subtract the background signal and/or normalize the image by brightness for the entire photo/video shooting period. However, not all steps all obligatory for the initial image processing.

### Noise Reduction

The results in the experiment are achieved by increasing the color depth of the image to 32-bit and noise reduction by Gaussian blur filter as a preliminary image processing.

The original image is divided into two segments, which can be called SIGNAL and NOISE. The SIGNAL segment contains frames with detected NADH fluorescence, whereas the NOISE segment contains frames before or after exposure to excitation radiation in which NADH fluorescence is not detected.

The resulting NADH fluorescence intensity values are subjected to mathematical processing for subsequent formation of ischemic injury estimation with regard to their behavior over time. Within the framework of the present application, several approaches to such processing were tested, and the four most successful approaches are specifically discussed herein: Method No. 1; Method No. 2; Differential Equations (a method for approximating photobleaching curves by solving a system of differential equations); Artificial Neural Network (a method of classifying photobleaching curves using a neural network).

Processing according to Method No. 1 and Method No. 2 is carried out in two stages. In the first stage, for each pixel (area), the parameter characterizing the photobleaching intensity is calculated by bringing fluorescence intensity values over time to one figure (for all frames for one shooting). The second stage is performed to remove the influence of uneven illumination of the tissue by excitation radiation if required.

### Standard Deviation

The calculation of the photobleaching intensity parameters for pixels in order to describe a single digital value was carried out according to formula (1) below for both the SIGNAL segment (FIG. 8A) and the NOISE segment (FIG. 8B).

$${SD}_{xy} = \sqrt{\frac{1}{{z}_{max} - 1} {\sum }_{z = 1}^{{z}_{max}} {( {I}_{xy} - {\overline{I}}_{xy} )}^{2}} \qquad ( 1 )$$

where I_{xy} is signal intensity in the pixel with coordinate (x, y);
zₘₐₓ is the number of frames in the video with NADH fluorescence (maximum z value) in the SIGNAL and NOISE segments;
Ī_{xy} is the arithmetic average of I_{xy} over the entire video;
SD_{xy} is the standard deviation over all frames for the area with coordinates (x, y).

SD²_{photobleaching}=SD²_{signal}−SD²ₙₒᵢₛₑ, where SD_{signal} is the standard deviation of the signal for all frames in which NADH fluorescence is present, and SDₙₒᵢₛₑ is the average value of the standard deviation for those frames in which NADH fluorescence is absent.

For each pixel, a digital value is calculated according to formula (1) to form a two-dimensional map (two-dimensional data array) containing these values according to the coordinates (x, y).

Calculated photobleaching maps are stored in a digital format for further processing, use, and/or display, wherein for each pixel with (x, y), the value of SDphotobleaching (x, y) is saved (FIG. 8C).

If ischemic injury is evaluated in a large-sized area (e.g., an entire organ or part of an organ) where an uneven distribution of specific excitation power of illumination that reaches the surface of the organ is expected due to the geometry of the organ and/or the mutual displacement of the excitation source and the irradiated organ, as well as edge effects at the organ boundaries where illumination is significantly different due to the natural shape of the organ, the negative effects of these factors must be eliminated.

To apply Method No. 1 and Method No. 2, it is necessary to calculate the minimum and maximum NADH fluorescence intensity values in each area (x, y) for all frames for SIGNAL and NOISE segments.

### Calculation of Minimum Intensities

The I′_{xy} values of the minimum intensities are calculated at each point (x, y) of the source video according to the formula (2):

$${I}_{xy}^{′} = {min}_{z} {I}_{xy} ; \qquad ( 2 )$$

where I_{xy} is signal intensity in the pixel with coordinate (x,y) and
min_{z} I_{xy}—is the minimum of this value over all z from 1 to zₘₐₓ for SIGNAL (FIG. 8D) and NOISE (FIG. 8E) video segments.

Then, the background I_{MIN_noise} value is subtracted from the I_{MIN_signal} value.

The resulting minimum intensities are stored digitally for further processing, use, and/or display (FIG. 8F).

The dimensionality of the minimum intensity map is the same as that of the NADH photobleaching map.

The I′″_{xy} values of the maximum intensities are calculated at each point (x, y) of the source video using the formula (3):

$${I}_{xy}^{′′′} = {max}_{z} {I}_{xy} ; \qquad ( 3 )$$

where I_{xy} is signal intensity in the pixel with coordinate (x,y) and
max_{z} I_{xy} is the maximum of this value over all z from 1 to zₘₐₓ (for SIGNAL and NOISE video segments).

### Method No. 1-Metric 1 (4th Degree)

The correction of NADH photobleaching maps to minimum intensity maps is performed for regions with matching coordinates (x, y). For each such region, the value A (x, y) is calculated by dividing two numbers using formula (4):
SD_{photobleaching}(x, y) is the value for the “photobleaching” map in the (x,y) area by
(I_{MIN}^{\hat{ }4})/k (FIG. 8G), where I_{MIN}′ is the value for the “minimum intensity” map in the (x, y) region, and k is the scale factor varying depending on the experimental conditions. In the series of experiments described, k=10^{\hat{ }10}.

$$A ( x , y ) = \frac{S {D}_{photobleaching} ( x , y )}{( {I}_{MIN} ’ \hat{} 4 / k )} \qquad ( 4 )$$

The A (x,y) values reflect the NADH activity in each pixel of the sample, i.e., they are proportional to the activity of the ischemic injury biomarker. The A (x,y) map maps ischemic tissue/organ injury.

Updating values in the NADH minimum intensity map from the NADH photobleaching map is performed to eliminate the effect of uneven distribution of the specific power of excitation radiation over the surface of the organ (FIG. 8H).

The “standard deviation” method is an alternative approach.

FIG. 9A shows the photobleaching curves for one region of the heart for two consecutive exposures to the excitation source (Source 1). It can be seen that the second graph is lower than the first—the concentration of NADH molecules is lower because the concentration of NADH has not recovered to its original level due to the short interval between the two exposures.

The attached figures also show maps of processing steps for two consecutive recordings: maximum and minimum intensity maps (FIGS. 9B and 9E), standard deviation maps (FIGS. 9F and 9G), ischemia map based on Method No. 1 (metric A=SD/(Iₘᵢₙ)⁴) with a gray scale from 0 to 2.5 (FIGS. 9H and 9I), ischemia map based on Method No. 2 (metric (SD/Iₘᵢₙ) 0.125/Imax) with a gray scale from 0.5 to 1.5 (FIGS. 9J and 9K).

Method No. 2-Metric 2 (degree ⅛)

Along with computing SD_{xy} by formula (1) and I′xy by formula (2), I′″_{xy} is calculated by formula (3) and I″_{xy} by formula (5). The value of A_{(x,y)} for Method No. 2 is computed by formula (6).

$${I}_{xy}^{′′} = {( \frac{{SD}_{xy}}{{I}_{xy}^{′}} )}^{\frac{1}{8}} \qquad ( 5 )$$

$${A ’}_{( x , y )} = \frac{{I}_{xy}^{′′}}{{I}_{xy}^{′′′}} \qquad ( 6 )$$

The A′(x,y) values reflect NADH activity in each pixel of the sample, i.e., they are proportional to the activity of the ischemic damage biomarker. Map A′ (x,y) is a map of ischemic tissue/organ damage.

A comparison between the processing and results for Method No. 1 and Method No. 2 is shown in FIG. 10.

### System of Differential Equations

For interpreting the photobleaching signal, the curves can be approximated by solving a system of differential equations describing the chemical kinetics of the NADH photobleaching reaction.

In living tissue, the NADH molecule can change into other forms (NAD or radical NAD*) for various reasons. Photobleaching is the transition of NADH molecule to NAD molecule under the influence of ultraviolet light with the formation of an H⁺ proton and an electron [Joubert F. et al. NADH enzyme-dependent fluorescence recovery after photobleaching (ED-FRAP): applications to enzyme and mitochondrial reaction kinetics, in vitro/Biophysical journal.—2004.—T. 86.—No. 1.—C. 629-645].

$$N A D H \to N A {D}^{*} + {H}^{+} + {e}_{aq}^{-} \qquad ( 7 )$$

To estimate the concentration of NADH in the tissue by approximating the experimental curve, a mathematical model of the chemical kinetics of the reaction of NADH photobleaching was compiled. The model calculates the change of NADH fluorescence signal during NADH photobleaching and reduction based on the relations given in [Joubert F. et al. NADH enzyme-dependent fluorescence recovery after photobleaching (ED-FRAP): applications to enzyme and mitochondrial reaction kinetics, in vitro/Biophysical journal.—2004.—T. 86.—No. 1.—C. 629-645]. The equations used in the mathematical modeling are as follows:

$$\frac{dNADH}{dt} = - A \times {N}^{λ} \times NADH + V ( NADH , NAD , … ) \qquad ( 8 )$$

$$NADH + NAD = const \qquad ( 9 )$$

$${V}_{i} = \frac{( \frac{{V}_{maxf}}{{K}_{NAD}} ) ( [ {NAD}_{0} ] - \frac{[ {NADH}_{0} ] [ {B}_{i} ]}{[ {A}_{i} ] {K}_{eq}} )}{1 + \frac{[ {NAD}_{0} ]}{{K}_{NAD}} + \frac{[ {NADH}_{0} ]}{{K}_{NADH}}} , \qquad ( 10 )$$

$$\begin{matrix}[ {A}_{i} ] - concentration A \\ [ {B}_{i} ] - concentration B\end{matrix}$$

$${K}_{eq} = [ {NADH}_{i} ] [ {B}_{i} ] / [ {NAD}_{i} ] [ {A}_{i} ] . \qquad ( 11 )$$

V_{maxf} is the value responsible for NADH molecule reduction λ is the degree of dependence of the NADH photobleaching rate on the excitation radiation power
NADHₚ is the concentration of the photobleached NADH form.

$$\begin{matrix}{C}_{1} = \frac{( \frac{{V}_{maxf}}{{K}_{NAD}} ) ( α + \frac{[ {NAD}_{i} ]}{[ {NADH}_{i} ]} )}{( 1 + \frac{[ {NAD}_{i} ]}{{K}_{NAD}} + \frac{[ {NADH}_{i} ]}{{K}_{NADH}} )} \\ {C}_{2} = \frac{( \frac{α}{{K}_{NAD}} - \frac{1}{{K}_{NADH}} )}{( 1 + \frac{[ {NAD}_{i} ]}{{K}_{NAD}} + \frac{[ {NADH}_{i} ]}{{K}_{NADH}} )}\end{matrix} . \qquad ( 12 )$$

$$Recov = \frac{C 1 \times {NADH}_{p}}{( 1 + C 2 \times {NADH}_{p} )} \qquad ( 13 )$$

$$Photo = A \times {NADH}_{p} \times {N}^{4} \qquad ( 14 )$$

$$Δ {NADH}_{p} - Recov , \qquad ( 15 )$$

where NAD and NADH are concentrations of NAD and NADH molecules;
Recov is an increment in NADH concentration for the analyzed time interval (per iteration, e.g., 1 shot);
Photo is a decrease in NADH concentration for the analyzed time interval (per iteration, e.g., 1 shot);
K_{NAD} is the reaction rate constant for the conversion of NAD to NADH;
K_{NADH} is the reaction rate constant for the conversion of NADH to NAD;
N is the power of illumination by excitation radiation;
A is the amplitude coefficient determining the type of the photobleaching curve (the rate of decrease in NADH concentration);
V is the coefficient of NADH concentration recovery rate;
λ is the degree of dependence of the NADH photobleaching rate on the excitation radiation power (in the described examples, it is assumed to be 2).

Alpha (or α) is a fraction (takes values from 0 to 1) of NADH, which is irreversibly converted to NAD by photobleaching, i.e., it is not reduced to NADH and does not glow when exposed to subsequent excitation radiation.

In the experiments, we achieved the result by using software written in Python to find numerical solutions for each of the parameters determining the shape of the approximated curve described by a system of differential equations given above.

We fixed the parameters Knad, Knadh, A, and alpha to reduce the computational cost. We set the range limits for fitting the parameters V and N, which were selected programmatically in a fixed range.

The brightness value of the excited radiation (I, fluorescence intensity) of the photobleaching curve is taken to be NADH×N and the NADH concentration is calculated using the formula (16) below:

$$NADH = {I}_{max} / N \qquad ( 16 )$$

FIG. 11 illustrates the operation of the mathematical model. For each photobleaching curve under study, the solution to the problem of approximation of kinetics curves for NADH photobleaching chemical reaction is selected by fitting the values of parameters V and N, while the other parameters are either fixed or obtained by the formulas laid down in the mathematical model.

The curve approximation method is described as follows.

To reduce the computational cost of further approximation of the model curve, the resulting experimental curve can be preprocessed: cleaned of noise, normalized to some value (e.g., the initial NADH fluorescence intensity), etc. This step is desirable but optional. In the experiments, the results were achieved through preprocessing the curves by Gaussian blurring with values of 0.5 in space and 4 in time and also by normalizing the NADH fluorescence intensity in the sequence of frames by the fluorescence intensity of its brightest frame (Imax).

Parameters N and V for numerical solution of the system of differential equations are iteratively chosen to match the model curve with the experimental curve within the selected accuracy not exceeding a pre-selected threshold value determined using the least square method (LSM). Other options for determining the accuracy of curve matching (comparison of integrals under curves, etc.) are also acceptable.

The solution to the problem of approximating the curves of the kinetics of chemical reactions of NADH photobleaching yields a set of parameters N and V for each curve. The sought parameter is the NADH concentration calculated for each curve by formula (15).

FIG. 12 shows an example of applying the above curve approximation method to interpret photobleaching curves. Two consecutive images of the rat heart following prolonged preservation were processed using said method. Parameters for the experiment were chosen such that the concentration of NADH in the irradiated region was the same for two shoots.

Radiation was excited by Source 1 at 60% and 100% of its maximum power (diode with 365±20 nm emission, specific power at 100% corresponds to approximately 500 mW/cm²).

The result was obtained as follows. To cut the computational cost of the problem, the frame size was preliminarily reduced to 16×16 pixels. For shooting at 60% excitation radiation (FIG. 12A) in a selected pixel, a set of parameters (N and V) of the system of differential equations of the mathematical model approximating the model curve was chosen. The resulting parameter V was used to approximate the curves for the remaining processed imaging pixels at 60% excitation radiation (FIG. 12A). Only the parameter N was chosen by solving differential equations until the difference between the NADH photobleaching curve normalized to the initial value and the model curve (calculated by least squares) reached the value 0.025. Similarly, all curves of the second image were approximated (at 100% excitation power) (FIG. 12B). Knowing the initial fluorescence intensity in each imaging pixel, the value of NADH concentration was determined by formula (16) after fitting the parameter N. The results are shown in FIG. 12C and

FIG. 12D. Unlike NADH fluorescence intensity maps, NADH concentration maps are similar for both shoots, which confirms the practical applicability and operability of the method of approximating photobleaching curves to determine and visualize ischemic damage.

### Neural Network

Another way to interpret the NADH photobleaching signal may be to classify photobleaching curves using trained neural networks (“neuronets”) and other artificial intelligence techniques.

The method of classification of NADH photobleaching curves by neural networks can be divided into the following stages: choosing architecture and building a neural network; preparing a data set (photobleaching curve+its characteristic parameters) for training the neural network; training the neural network; using the neural network on experimental data.

In order to classify photobleaching curves, a type of neural network was applied that is suitable for describing “time series,” i.e., time-dependent processes, where each next value depends only on the previous one. Such networks are called recurrent. In the experiments, the neural network was implemented based on LSTM (Long Short-Term Memory) modules, a typical implementation for recurrent neural networks. In the experiments, an artificial neural network based on LSTM modules was a program code aimed to convert photobleaching curves (obtained from a sequence of data including frame number and fluorescence brightness value in the processed region) into required parameters and values uniquely characterizing the photobleaching curve, including the parameter characterizing NADH concentration for the photobleaching curve processed. In the experiments, the neural network program code was written in Python and converted to a “.pth” file. It may use other programming languages and corresponding file formats in other variants.

Optionally, the photobleaching curve can be preprocessed before being processed by the neural network.

Training a neural network requires a large number of illustrative examples of solving the classification problem, for example, sets of “photobleaching curve+its characteristic parameters,” where the parameters are a set of values uniquely characterizing the photobleaching curve (and thus the tissue state). In the experiments performed, it was challenging to measure tissue metabolic parameters to train the model; therefore, we used curves obtained by solving a system of differential equations describing the chemical kinetics of the NADH photobleaching reaction.

With the help of a mathematical model of the kinetics of the NADH photobleaching reaction, it was shown that a single fluorescence curve corresponds to a single combination of three parameters: the fluorescence intensity change value, the excitation radiation power, and the strength of the reducing NADH enzyme.

In order to train the neural network, a sample of 30,000 sets of “photobleaching curves and their parameters” obtained using the system of differential equations described above was compiled. Four parameters were selected for variation: N (exposure power); α (alpha)—relative amount of NADH irreversibly transferred to NAD, not recovered further; V—amplitude factor for NADH recovery; and+I—NADH fluorescence intensity at the initial point of the photobleaching curve.

FIG. 13 shows graphs of curve classification error dependencies on the training stage obtained from the neural network training. The error decreases monotonically with training. The horizontal axis shows the iteration of training, i.e., the number of stages (so-called “epochs”) spent on training. The vertical axis shows the value of the classification error of the photobleaching curve by the neural network. The errors obtained by each of the three ways of evaluating the learning success—(A) Evaluation loss analysis, (B) Train loss analysis, (C) Evaluation score analysis—monotonically decrease with reaching “saturation,” i.e., the minimal error value for the network with the chosen architecture.

FIG. 14 and Table 1 below demonstrate experimental data used to test the neural network performance. NADH photobleaching plots normalized to the initial value and obtained using rat hearts for illumination at 80%, 90%, and 100% of the maximum source power were classified using a trained neural network. For this purpose, the experimental data were preprocessed with a Gaussian filter to eliminate noise. At the same time, the design of the experiment helped to ensure that all parameters, except the illumination power, in the analyzed images were the same. As can be seen, the results of using a neural network with an accuracy to the trained neural network error of 9% coincide with the experimental, which indicates the successful training of the neural network.

TABLE 1
Neural network performance result

| | Relative | | | | | | | | |
|---|---|---|---|---|---|---|---|---|---|
| | exposure | Neural | Neural | | | | | | |
| | power (fraction | network | network | | | | | | |
| Point | of 500 | performance: | performance: | Relative value | Relative value | | | | |
| No. | mW/cm²), % | N, c.u. | V, c.u. | of N, % | of V, % | K_{NAD} | K_{NADH} | A | α |
| 0 | 100 | 0.002823 | 0.117374 | 100 | 100 | 10⁻⁴ | 10⁻⁴ | 10⁷ | 0.95 |
| 1 | 90 | 0.002642 | 0.124012 | 93.6 | 106 | 10⁻⁴ | 10⁻⁴ | 10⁷ | 0.95 |
| 2 | 80 | 0.002271 | 0.132956 | 80.4 | 112.8 | 10⁻⁴ | 10⁻⁴ | 10⁷ | 0.95 |

Neural network performance result. Analysis of the experimental curves yielded a relative exposure power of 100%, 93.6%, and 80.4%, which fits within the limits of acceptable errors typical for a successfully trained neural network.

A comparison of processing results in one experiment by all four processing methods described is shown in FIG. 15. The results of three consecutive illuminations of the same region of the human heart were obtained during cardiac surgery of aortocoronary bypass surgery on a stopped heart. As can be seen, all four methods adequately reflected organ ischemia, which was minimal immediately after cardiac arrest, maximal at the end of the period of aortic constriction, and average immediately after the restoration of blood flow in the organ.

### EXAMPLES

Experiments illustrating embodiments of the invention have been performed in considerable numbers on cells, organs, parts, and tissues, including the heart, liver, kidney, spleen, and muscle tissue.

### Example 1—Experiments on Laboratory Rat Hearts

A group of eight rat heart experiments was performed under isolated preserved heart conditions with periodic monitoring of organ ischemia for 6 hours. The experimental setup consisted of two main parts: (I) a preserving circuit and (II) a device for non-invasive monitoring of the organ condition (FIG. 5A). The experiments were performed on rat hearts taken from Sprague Dawley rats after euthanasia with chloroform. After rapid extraction, the heart was placed in a chamber and connected to a circuit for preservation. Heartbeats were recorded for 5 minutes, then stopped with a cardioplegic solution. Two cardioplegic solutions were used to stop the heart in a series of experiments (n=8): blood (n=4) and crystalloid (n=4). For blood cardioplegia, cardioplegia was added to the thermostatic reservoir in a single dose to achieve a ratio of blood cardioplegia: Tirode=1:4. In crystalloid cardioplegia, full replacement of Tyrode's solution with crystalloid cardioplegia was performed. After cardiac arrest, perfusion with the cardioplegic solution was continued for up to 6 hours while maintaining the specified temperature regime. NADH fluorescence was recorded periodically (approximately every 20 minutes), and a map of ischemic damage for the perfused organ was constructed.

Saving for further analysis and online processing of NADH fluorescence recordings (250 mW UV power 500 mW/cm², spot diameter 2 cm, pulse duration 10 seconds, video sampling rate 50 fps, resolution 380×351 px) was performed using a computer combined with a PCO.1200hs camera. Two methods were used for operational processing: Method No. 1 in the Python environment and a macro in the Image J software.

When the scheduled perfusion observation time (6 hours) was over, a complete replacement of perfusate with fresh oxygenated Tyrode's solution was performed. Then, within 30 minutes, the resumption of cardiac mechanical contractions (or their absence) was recorded using a PCO-camera; NADH fluorescence was also recorded to determine the final localization of ischemic foci, and a map of ischemic organ damage was constructed at the end of the experiment. If no mechanical activity of the heart was recorded after 30 minutes of perfusion with oxygenated Tyrode's solution, the susceptibility of the heart to electrode and mechanical stimulation was checked. At the end of the experiment, the heart was thoroughly washed and fixed in PFA (paraformaldehyde) for further studies.

FIG. 16 and Table 2 show the resulting NADH concentrations. It can be seen that if the maximum value of NADH concentration calculated with Method No. 1 is 2 times (200%) higher than the initial value, the heart will not contract at the end of the experiment.

TABLE 2
Values of NADH concentration in experiments with rat hearts

| | | 1 hour | | 2 hours | | 3 hours | | 4 hours | | 5 hours | | 6 hours | | | |
|---|---|---|---|---|---|---|---|---|---|---|---|---|---|---|---|
| | | 1 | 2 | 3 | 4 | 5 | 6 | 7 | 8 | 9 | 10 | 11 | 12 | Max % | |
| | | Reference NADH concentration from Method No. 1 - value (% of | | | | | | | | | | | | of | |
| Experiment | Perfusion | baseline) | | | | | | | | | | | | baseline | Result* |
| No. 1 | Crystalloid | 100 | 167 | 224 | 271 | 144 | 113 | 103 | 216 | — | — | — | — | 271 | − |
| | cardioplegia | | | | | | | | | | | | | | |
| No. 2 | Blood | 100 | — | 99 | 103 | — | 88 | — | 82 | — | — | 142 | 65 | 142 | + |
| | cardioplegia | | | | | | | | | | | | | | |
| No. 3 | Blood | 100 | 297 | 245 | 244 | 273 | 292 | 229 | 284 | 172 | 175 | 196 | 208 | 297 | − |
| | cardioplegia | | | | | | | | | | | | | | |
| No. 4 | Blood | 100 | 75 | — | 50 | 47 | 49 | 30 | 22 | — | 25 | 25 | 27 | 100 | + |
| | cardioplegia | | | | | | | | | | | | | | |
| No. 5 | Crystalloid | 100 | 49 | 55 | 68 | 67 | 93 | 84 | 77 | — | — | — | — | 100 | + |
| | cardioplegia | | | | | | | | | | | | | | |
| No. 6 | Crystalloid | 100 | 85 | 207 | 152 | 128 | 151 | 161 | 156 | 202 | 159 | 185 | 196 | 207 | − |
| | cardioplegia | | | | | | | | | | | | | | |
| No. 7 | Crystalloid | 100 | 99 | 180 | 142 | 156 | 122 | 79 | 170 | 150 | 146 | 114 | 113 | 180 | + |
| | cardioplegia | | | | | | | | | | | | | | |
| No. 8 | Blood | 101 | 103 | 117 | 77 | 47 | 46 | 50 | 55 | 54 | 47 | 47 | 40 | 117 | + |
| | cardioplegia | | | | | | | | | | | | | | |

*(−) the heart did not retain contractility, or there were rhythm abnormalities; (+) contractile activity at the end of the experiment

Reference NADH concentration maps obtained using Source 1 by Method No. 1 are shown in FIG. 17.

### Example 2-Experiments on Rat Liver, Kidney, Spleen, and Muscle Tissue

Here, the difference between the experimental procedure and Example 1 was that the organ and tissue were not perfused with solutions but were preserved in a crystalloid cardioplegic solution or PBS (phosphate-buffered saline) solution for 24 hours for the liver and muscle tissue and 4 hours for the kidney and spleen.

The resulting maps of ischemic damage (FIG. 18, FIG. 19, and FIG. 20) demonstrate that the ischemic assessment method using the NADH biomarker fundamentally applies to these organs.

FIG. 18 demonstrates different conditions of the liver when the organ is preserved in two different solutions. The scale for these drawings is the same and indicates approximate equality of initial ischemia values (immediately after liver isolation and placing the liver pieces in the respective solutions) and difference in ischemia parameters at the final time point (in the case of PBS, the ischemia is much more severe). After extraction, the liver was divided into two parts: one part was placed into a PBS solution (FIGS. 18A and 18B) and the other into a crystalloid cardioplegic solution (FIGS. 18C, 18D, and 18E). This experiment demonstrates adequate liver preservation using cold crystalloid cardioplegia: low solution temperature (+4 to +10 degrees) reduces the metabolic requirement of the tissue without disturbing the enzyme activity. The optical properties of the liver surface are different from those of the heart. The same applies to the other organs: with similar exposure to ultraviolet radiation, the amount of absorbed ultraviolet radiation differs for different tissue types. This experiment demonstrates that the absence of cold crystalloid cardioplegia (instead of which an isotonic glucose-free PBS buffer solution was used) cannot sufficiently reduce the metabolic demand of the tissue, which leads to severe ischemia of the organ.

Earlier experiments (M.Kay) confirm the stable performance of the enzyme dehydrogenase during multiple photobleaching of NADH in cardiac tissue: after about 100 seconds, the fluorescence intensity of NADH molecules returns to the initial values (before photobleaching). Judging by our experiment with the liver, the enzyme activity is also sufficient to restore NADH, which makes it possible to obtain stable visualization of ischemia foci. Otherwise, if the dehydrogenase does not work intensively enough and does not recover NADH, the ischemia intensity map values would decrease dramatically with each following recording.

The kidney in FIG. 19 was preserved in a hypothermic crystalloid cardioplegic solution. Neither significant changes in the intensity of the ischemia map values nor their decrease were observed (FIG. 19B). The latter indicates the adequate work of the enzyme in the conditions of preservation of the organ. This may indicate that the metabolic needs of the kidney are comparable to those of the liver.

The spleen was preserved in a hypothermic crystalloid cardioplegic solution. Visualization of NADH in the spleen was complicated by its optical properties and low concentration of NADH molecules: the NADH fluorescence signal was significantly weaker than that of other tissues at approximately equal irradiation intensity. In this case, photobleaching was barely separable from random intensity changes (recording noise). Hence, an unambiguous comparison of ischemia map intensity and the extent of organ ischemia is difficult (calculation errors are comparable to or exceed changes in the required value). This suggests that NADH imaging in tissues with low metabolism requires higher irradiation exposure.

The muscle tissue in FIG. 20 was preserved in a hypothermic crystalloid cardioplegic solution under the same conditions and for the same amount of time as the liver. A comparison of the liver in FIG. 18 and muscle tissue in FIG. 20 ischemia maps show a significantly greater extent of ischemia in muscle tissue. This may indicate that the metabolic demands of muscle tissue were significantly higher than those of the liver.

### Example 3—Experiment with a Monolayer of Human Cardiomyocytes

The optical mapping technique makes it possible to evaluate cell functionality and unambiguously separate normally functioning tissue (contracting cardiomyocytes) from an injured one. Before the experiment, the monolayer of cardiomyocytes was stripped using optical mapping with FluoVolt potential-dependent fluorescent dye to ensure their full contractile capacity (optical mapping, FIG. 21A, then preserved in a crystalloid cardioplegic solution for 4 hours, after which the solution was replaced with a growth medium and maintained in the incubator for another 7 days. After 7 days, optical mapping was performed again to determine which cardiomyocytes retained contractility (FIG. 21B), and the reference NADH concentration was determined using Method No. 1, FIG. 21C. Overlaying the reference NADH concentration map of cardiomyocyte contractility, we found that the NADH value for areas where cardiomyocytes completely retained contractility was 85±7 units. For areas where cardiomyocytes lost contractility, the NADH value was 121±3 units. A map of the reference NADH concentration, colored on a scale with account taken of the above experimental values, is shown in FIG. 21D.

Using optical mapping in whole hearts is impossible in practice. To obtain quantitative values for the extent of ischemia (reference NADH concentration) for three-dimensional cardiac tissue, the model of the chemical reaction of photobleaching kinetics was extended from a 2-dimensional monolayer model to a 3-dimensional tissue model, and using the extended model, a staining (scale) map of ischemia in rat heart experiment 1 was obtained. FIG. 21E Using optical mapping in whole hearts is impossible in practice. In order to obtain quantitative values for the extent of ischemia (reference NADH concentration) for three-dimensional cardiac tissue, the model of the chemical reaction of photobleaching kinetics was extended from a 2-dimensional monolayer model to a 3-dimensional tissue model, and using the extended model, a staining (scale) map of ischemia in rat heart experiment 1 was obtained. FIG. 21E and FIG. 21F show a reference NADH concentration maps of the rat heart plotted against the ischemia score scale obtained by mathematical modeling of the chemical reaction kinetics of photobleaching and ischemic injury modeling of 3D cardiomyocyte layers.

### Example 4—Experiments on the Heart of Large Animals (Pigs)

The experimental framework is as follows. The pig is put into narcotic sleep, and the heart is extracted and placed in a medical box for preservation. Regularly (every 30 or 60 minutes), imaging of the organ or its part is performed to monitor the ischemic damage. Depending on the condition of the organ and the results of NADH monitoring, a decision s made to change the preservation parameters. The experiment is completed either with the stage of cardiac rhythm and pump function recovery or with the stage of intentional ischemia with monitoring of NADH state in the tissue. The duration of the experiments varied from 9 to 24 hours. Shown here are experiment 6, with a time length of 14 hours, and experiment 13, with a time length of 17 hours.

FIG. 22A illustrates the processing of Porcine Heart Preservation Experiment No. 6 using Method Nos. 1 and 2. As a result of this experiment, the heart did not survive preservation or regain contractile activity. The graph at the end of the experiment shows a significant increase in NADH concentration: about 200 times the result of Method No. 1 processing and about 7 times the result of Method No. 2 processing. Following an increase in NADH concentration, it decreased according to the results of processing by both approaches, which may indicate organ death, while a decrease in NADH concentration indicates it is burning out without subsequent recovery due to cardiomyocyte death. In the experiment, Source 1 was used for NADH monitoring.

FIG. 22B shows a graph of heart ischemia monitoring by Source 1 in Experiment 13 aimed at preserving the porcine heart after processing with Method Nos. 1 and 2. As a result of the experiment, the heart recovered its contractile activity. The graph shows fluctuations of NADH concentration during the experiment within acceptable limits without its fatal growth leading to the organ's death. Fluctuations in NADH concentration are explained by changes in the organ preservation regimen and the administration of drugs.

FIG. 22C shows a graph of cardiac ischemia monitoring by Source 2 in Experiment 13 after processing with Method Nos. 1 and 2. The organ's exposure by Source 2 was performed with a time shift of 30 minutes relative to the exposures by Source 1. This explains the slightly different nature of the curves of Method Nos. 1 and 2. The fluctuations in NADH concentration, which are also explained by the changes in the preservation mode, were within the range of tolerance.

FIG. 23 shows maps of cardiac ischemia in Experiment 13 obtained using Source 2 after processing with Method Nos. 1 and 2: A and D at the beginning, B and E in the middle, and C and F at the end of the experiment. The maps show some nonfatal increase in ischemia toward the end of the experiment, which is consistent with the ischemia monitoring plots shown in FIG. 23 (B and C).

### Example 5—Approbation of the Invention on the Human Heart

The approbation was planned in order to confirm the operability of the method and device on the human heart in the conditions of a real cardiac surgery of aortocoronary bypass surgery (CABG).

Three images were taken: immediately after cardiac arrest, immediately before restoration of cardiac blood flow, and immediately after restoration of cardiac blood flow. FIG. 15 shows corresponding ischemia maps obtained using Method Nos. 1 and 2. Both methods demonstrate the initial state of the heart at the beginning of the operation immediately after (cardiac arrest for CABG surgery (FIG. 15 (Method No. 1, Image 1), (Method No. 2, Image 1)), increase in ischemia (increase in bright areas on the image) during surgery (FIG. 15 (Method No. 1, Image 2), (Method No. 2, Image 2)), and improvement after the restoration of blood flow (FIG. 15 (Method No. 1, Image 3), (Method No. 2, Image 3)). Two other processing methods, the method of approximating the photobleaching curves by the solution of a system of differential equations and the method of classifying the photobleaching curves by a neural network, show the same results.

What claimed is:

1. A method for assessing ischemic injury of the tissue, comprising:
(a) excitation of NADH;
(b) recording NADH fluorescence; and
(c) evaluating fluorescence intensity values over a duration of time.

2. The method according to claim 1,
wherein the evaluating step includes subtracting noise is from the recorded fluorescence.

3. The method according to claim 1,
wherein the evaluating step includes determining a value for the standard deviation of the intensity of the excited fluorescence.

4. The method according to claim 3,
wherein the evaluating step includes determining a minimum intensity of excited fluorescence raised to the fourth power, and
dividing the value for the standard deviation of intensity of excited fluorescence by the value of the minimum intensity of excited fluorescence raised to the fourth power.

5. The method according to claim 3,
wherein the evaluating step is includes determining a minimum intensity of excited fluorescence and a maximum value of excited fluorescence, and
dividing the value for the standard deviation of intensity of the excited fluorescence by the value of the minimum intensity of excited fluorescence and raising this quotient to the ⅛th power with subsequent dividing by the maximum value of excited fluorescence.

6. The method according to claim 1,
wherein the evaluating step includes approximating photobleaching intensity curves through solving a system of differential equations describing the kinetic model of photobleaching to determine NADH concentration, excitation radiation power, and the strength of the NADH reducing enzyme.

7. The method according to claim 1,
wherein the evaluating step includes classifying photobleaching intensity curves using a neural network and recovering for each curve a unique set of variables, including NADH concentration, excitatory radiation power, and the strength of the NADH reducing enzyme.

8. The method according to claim 1,
wherein the excitation of NADH comprises exposing the tissue to excitation radiation having a wavelength of 365 nm.

9. The method according to claim 1,
wherein recording NADH fluorescence comprises recording radiation having a wavelength of 460 nm.

10. The method according to claim 1,
wherein the ratio of time and the specific power of the excitation radiation that reaches the surface of object under study must be sufficient to obtain a photobleaching curve.

11. The method according to claim 1,
wherein the method steps (a)-(c) are performed repeatedly over time to track the dynamics of the emergence and progression of ischemic damage areas.

12. A device for assessing ischemic injury of the tissue using the method according to claim 1, said device comprising
a unit for excitation of NADH fluorescence comprising a UV-LED and a lens;
a unit for recording NADH fluorescence comprising an optical filter and a camera; and
a unit for processing fluorescence intensity values over a duration of time comprising a computer.

13. The device according to claim 12,
wherein the computer is configured to subtract noise is from the recorded fluorescence.

14. The device according to claim 12,
wherein the computer is configured to determine a value for the standard deviation of the intensity of the excited fluorescence.

15. The device according to claim 14,
wherein the computer is configured to determine a minimum intensity of excited fluorescence raised to the fourth power, and
divide the value for the standard deviation of intensity of excited fluorescence by the value of the minimum intensity of excited fluorescence raised to the fourth power.

16. The device according to claim 14,
wherein the computer is configured to determine a minimum intensity of excited fluorescence and a maximum value of excited fluorescence, and
divide the value for the standard deviation of intensity of the excited fluorescence by the value of the minimum intensity of excited fluorescence and raising this quotient to the ⅛th power with subsequent dividing by the maximum value of excited fluorescence.

17. The device according to claim 12,
wherein the computer is configured to approximate photobleaching intensity curves through solving a system of differential equations describing the kinetic model of photobleaching to determine NADH concentration, excitation radiation power, and the strength of the NADH reducing enzyme.

18. The device according to claim 12,
wherein the computer is configured to classify photobleaching intensity curves using a neural network and recovering for each curve a unique set of variables, including NADH concentration, excitatory radiation power, and the strength of the NADH reducing enzyme.

19. The device according to claim 12,
wherein the UV-LED is configured to emit an excitation radiation having a wavelength of 365 nm.

20. The device according to claim 12,
wherein the optical filter has a bandpass comprising a wavelength of 460 nm.

21. The device according to claim 12,
wherein the computer is configured to extract NADH fluorescence at a wavelength of 460 nm before clearing the digital fluorescence waveform from noise.

22. The device according to claim 12,
wherein the ratio of time and specific power of excitation radiation that reaches the surface of object under study must be sufficient to obtain data for constructing a map of ischemic injury of the organ.

23. The device according to claim 12,
wherein the UV-LED is configured to emit a specific power of ultraviolet radiation for NADH excitation between 1-50 mJ/mm².

24. The device according to claim 12,
wherein the camera is configured to record NADH fluorescence by photographing at 50 frames per second with a resolution of at least 512 pixels by at least 512 pixels.
